(19) United States
(12) Patent Application Publication
(10) Pub. No.: US 2015/0267178 A1
(43) Pub. Date: Sep. 24, 2015

(54) PROTEIN HAVING FLAVIN ADENINE DINUCLEOTIDE-DEPENDENT GLUCOSE DEHYDROGENASE ACTIVITY

(71) Applicant: NATIONAL INSTITUTE OF ADVANCED INDUSTRIAL SCIENCE AND TECHNOLOGY, Tokyo (JP)

(72) Inventors: Kazumichi Ozawa, Tsukuba-shi (JP); Noriko Sasaki, Tsukuba-shi (JP); Atsunori Hiratsuka, Tsukuba-shi (JP); Kenji Yokoyama, Tsukuba-shi (JP); Nao Kinoshita, Tsukuba-shi (JP); Hideaki Ooe, Nagaokakyo-shi (JP); Jun Takagi, Nagaokakyo-shi (JP); Koji Tanaka, Nagaokaky-shi (JP); Katsushige Kotani, Nagaokaky-shi (JP)

(21) Appl. No.: 14/425,837

(22) PCT Filed: Sep. 9, 2013
(86) PCT No.: PCT/JP2013/074199

§ 371 Date: Jun. 3, 2015

(30) Foreign Application Priority Data
Sep. 18, 2012 (JP) 2012-204507
May 29, 2013 (JP) 2013-113074

(51) Int. Cl. C12N 9/04; C12Q 1/54

(52) U.S. Cl. CPC C12N 9/0006; C12Q 1/54; C12Y 101/9901; G01N 2333/904

(57) ABSTRACT

The present invention provides a protein derived from a thermophilic filamentous fungus having flavin adenine dinucleotide-dependent glucose dehydrogenase activity. The present invention provides an enzyme for glucose measurement that has better thermal stability than that of conventional enzymes while being unaffected by dissolved oxygen, not requiring the addition of a coenzyme, and maintaining the excellent properties of FADGDH in terms of having excellent substrate specificity (particularly low reactivity with maltose).

### TECHNICAL FIELD

The present invention relates to a protein having flavin adenine dinucleotide-dependent glucose dehydrogenase activity, a gene encoding the protein, a recombinant vector including the gene, a transformant including the gene, a method for producing flavin adenine dinucleotide-dependent glucose dehydrogenase using the gene or the transformant, and a glucose sensor, a glucose concentration measuring method and a biofuel cell using the protein.

### BACKGROUND ART

Self-monitoring of blood glucose is important for a diabetic patient to grasp his/her own usual blood-sugar level and make use of it for therapy. A sensor for use in self-monitoring of blood glucose utilizes an enzyme that uses glucose as a substrate. Conventional examples of such an enzyme include glucose oxidase (EC 1.1.3.4). Glucose oxidase has been traditionally used as an enzyme for a blood glucose sensor because it has advantages of high specificity to glucose and excellent thermal stability.

In a blood glucose sensor utilizing glucose oxidase, glucose measurement is achieved in such a manner that an electron generated in the course of oxidizing glucose and converting it to D-glucono-1,5-lactone is delivered to an electrode via a mediator. However, since glucose oxidase tends to easily deliver the proton generated in the reaction to oxygen, there is a problem that dissolved oxygen influences on the measured value.

For avoiding such a problem, for example, nicotinamide adenine dinucleotide (NAD)-dependent or nicotinamide adenine dinucleotide phosphate (NADP)-dependent glucose dehydrogenase (GDH) (NAD(P)GDH: EC 1.1.1.47) or pyrrolo-quinoline quinone (PQQ)-dependent GDH (PQQGDH: EC 1.1.5.2) is used as an enzyme for a blood glucose sensor.

These are advantageous in that they are not influenced by dissolved oxygen. However, NAD(P)GDH has the disadvantages of poor stability, requiring addition of a coenzyme, and complicated measurement. PQQGDH has the disadvantages of poor substrate specificity, and poor reliability of measured value because it acts on saccharides other than glucose, such as maltose and lactose.

As an enzyme that overcomes these disadvantages, flavin adenine dinucleotide-dependent glucose dehydrogenase (FADGDH: EC 1.1.99.10) attracts attention. FADGDH is an enzyme that catalyzes the oxidation reaction from glucose to D-glucono-1,5-lactone using flavin adenine dinucleotide (FAD) as a coenzyme, and is found in normal temperature filamentous fungi such as Aspergillus oryzae and Aspergillus terreus. FADGDH has the advantages of not being influenced by dissolved oxygen, not requiring addition of any coenzyme, and having excellent substrate specificity.

FADGDH derived from Aspergillus oryzae or Aspergillus terreus is disclosed, for example, in PTD 1 (WO 2004/058958), PTD 2 (WO 2006/101239), PTD 3 (Japanese Patent Laying-Open No. 2008-178380), PTD 4 (Japanese Patent Laying-Open No. 2008-154573), PTD 5 (Japanese Patent Laying-Open No. 2008-154574), PTD 6 (Japanese Patent Laying-Open No. 2010-193913), PTD 7 (WO 2008/001903), PTD 8 (Japanese Patent Laying-Open No. 2011-55836), and PTD 9 (Japanese Patent Laying-Open No. 2011-217755).

Desirably, the enzyme for use in the glucose sensor has high thermal stability because the enzyme is sometimes subjected to a heat drying treatment in the step of preparing a reagent for the glucose sensor containing the enzyme, and it is also assumed that the environmental temperature during storage of the reagent for the glucose sensor rises to high temperature.

FADGDH derived from Aspergillus filamentous fungi described above has a certain degree of thermal stability if it is obtained from a wild-type strain by cultivation and purification. However, FADGDH produced by expression in recombinant E. coli that is best suited for mass production is proved to be greatly inferior in thermal stability to FADGDH obtained from a wild-type strain by cultivation and purification (PTD 10: WO2009/119728). The reduction in thermal stability is ascribable to the fact that a sugar chain is not added to the surface of an enzyme produced by recombinant E. coli.

For improving the thermal stability of FADGDH produced by expression in recombinant E. coli, for example, the method of genetically modifying the amino acid sequence is studied (PTD 10). The document discloses that when recombinant FADGDH is expressed in E. coli using a structural gene of FADGDH to which a specific mutation is added, FADGDH having thermal stability (stability of the GDH activity when FADGDH is heated at 50° C. for 15 minutes) comparative to that of FADGDH produced by a wild-type strain is obtained. However, since this is partial modification of FADGDH derived from Aspergillus filamentous fungus, it would not be easy to obtain FADGDH having more excellent thermal stability than FADGDH derived from a wild-type strain of Aspergillus filamentous fungus, and having properties of less likely to be deactivated under severer conditions.

### CITATION LIST

### Patent Document

### PTD 1: WO 2004/058958

### PTD 2: WO 2006/101239

### PTD 3: Japanese Patent Laying-Open No. 2008-178380

### PTD 4: Japanese Patent Laying-Open No, 2008-154573

### PTD 5: Japanese Patent Laying-Open No. 2008-154574

### PTD 6: Japanese Patent Laying-Open No. 2010-193913

### PTD 7: WO 2008/001903

### PTD 8: Japanese Patent Laying-Open No. 2011-55836

### PTD 9: Japanese Patent Laying-Open No. 2011-217755

### PTD 10: WO 2009/119728

### SUMMARY OF INVENTION

### Technical Problem

It is an object of the present invention to provide an enzyme for glucose measurement that has better thermal stability than that of conventional enzymes while being unaffected by dissolved oxygen, not requiring the addition of a coenzyme, and maintaining the excellent properties of FADGDH in terms of having excellent substrate specificity (particularly low reactivity with maltose).

### Solution to Problem

The present invention provides a protein derived from a thermophilic filamentous fungus, having flavin adenine dinucleotide-dependent glucose dehydrogenase activity.

The present invention also relates to a protein of any one of the following (a) to (d):

(a) a protein having an amino acid sequence represented by SEQ ID NO: 1 or 2,

(b) a protein having an amino acid sequence that is the amino acid sequence represented by SEQ ID NO: 1 or 2 from which a signal peptide is removed,

(c) a protein having an amino acid sequence that is the amino acid sequence of the protein of the above (a) or (b) from/to which one or several amino acid residues are deleted, substituted, added or inserted, and

(d) a protein having a homology of more than or equal to 70% with the amino acid sequence of the protein of any one of the above (a) to (c), and having flavin adenine dinucleotide-dependent glucose dehydrogenase activity.

Preferably, the protein is derived from a thermophilic filamentous fungus.

The present invention also relates to a protein having an amino acid sequence encoded by at least one base sequence selected from the group consisting of base sequences of SEQ ID NOs: 5 to 7, and base sequences complementary to base sequences of SEQ ID NOs: 8 and 9, and having flavin adenine dinucleotide-dependent glucose dehydrogenase activity. Preferably, the protein is derived from a thermophilic filamentous fungus.

Preferably, the thermophilic filamentous fungus is Talaromyces emersonii or Thermoascus crustaceus. Preferably, the above protein is a glycosylated protein.

The present invention also relates to a gene encoding the above protein.

The present invention also relates to a gene having a DNA of any one of the following (A) to (D):

(A) a DNA having a base sequence according to SEQ ID NO: 3 or 4,

(B) a DNA having a base sequence that is the base sequence of SEQ ID NO: 3 or 4 from which a base sequence encoding a signal peptide is removed,

(C) a DNA containing the base sequence of the DNA of (A) or (B) and an intron, and

(D) a DNA that hybridizes with a DNA having a base sequence complementary to the DNA of any one of (A) to (C) under a stringent condition, and encodes a protein having flavin adenine dinucleotide-dependent glucose dehydrogenase activity.

The present invention also relates to a recombinant vector including the gene.

The present invention also relates to a transformant including the gene.

The present invention also relates to a method for producing flavin adenine dinucleotide-dependent glucose dehydrogenase using the gene.

The present invention also relates to a method for producing flavin adenine dinucleotide-dependent glucose dehydrogenase using the transformant.

The present invention also relates to a glucose concentration measuring reagent containing the protein.

The present invention also relates to a glucose sensor using the protein.

The present invention also relates to a glucose concentration measuring method using the protein.

The present invention also relates to a biofuel cell using the protein.

### Advantageous Effects of Invention

In the present invention, since the protein (enzyme) derived from a thermophilic filamentous fungus and having FADGDH activity is found, it is possible to provide an enzyme for glucose measurement that has better thermal stability than that of conventional enzymes while being unaffected by dissolved oxygen, not requiring the addition of a coenzyme, and maintaining the excellent properties of FADGDH in terms of having excellent substrate specificity. Also, by using such an enzyme for glucose measurement, thermal inactivation of the enzyme during preparation or transportation of the glucose concentration measuring reagent or the glucose sensor is reduced, and it is possible to reduce the use amount of the enzyme and improve the measurement accuracy.

### BRIEF DESCRIPTION OF DRAWINGS

FIG. 1 is a photograph showing the result of the agarose gel electrophoresis of the degenerate PCR product of Ta. emersonii (NBRC31232) in Example 1.

FIG. 2 is a photograph showing the result of the agarose gel electrophoresis of the degenerate PCR product of Th. crustaceus (NBRC9129) in Example 1

FIG. 3 is a photograph showing the result of the agarose gel electrophoresis of the degenerate PCR product of Th. crustaceus (NBRC9816) in Example 1.

FIG. 4 is a chart showing the base sequence of the DNA fragment including the whole region of Ta. emersonii-derived FADGDH gene.

FIG. 5 is a schematic diagram showing the structure of the exon in the base sequence shown in FIG. 4.

FIG. 6 is a chart showing the base sequence of ORF of Ta. emersonii-derived FADGDH gene and an amino acid sequence analogized from the base sequence.

FIG. 7 is a chart showing homology between the amino acid sequences of FADGDH derived from thermophilic filamentous fungi (Ta. emersonii, Th. crustaceus) and the amino acid sequences of FADGDH derived from Aspergillus filamentous fungi.

FIG. 8 is a chart showing the base sequence of the DNA fragment including the whole region of Th. crustaceus-derived FADGDH gene.

FIG. 9 is a schematic diagram showing the structure of the exon in the base sequence shown in FIG. 8.

FIG. 10 is a chart showing the base sequence of ORF of Th. crustaceus-derived FADGDH gene and an amino acid sequence analogized from the base sequence.

FIG. 11 is a chart showing the result of SDS-PAGE in Example 4.

FIG. 12 is a chart showing the absorption spectrum regarding FADGDH derived from Ta. emersonii in Example 5.

FIG. 13 is a chart showing the absorption spectrum regarding FADGDH derived from Th. crustaceus in Example 5.

FIG. 14 is a chart showing the result of SDS-PAGE in Example 8.

FIG. 15 is a chart showing the absorption spectrum regarding FADGDH derived from Ta. emersonii in Example 9.

FIG. 16 is a chart showing the absorption spectrum regarding FADGDH derived from Th. crustaceus in Example 9.

FIG. 17 is a graph showing the residual activity percentage of FADGDH derived from Ta. emersonii in the pH stability evaluation in Example 10.

FIG. 18 is a graph showing the residual activity percentage of FADGDH derived from Th. crustaceus in the pH stability evaluation in Example 10.

FIG. 19 is a graph showing the relative activity of FADGDH derived from Ta. emersonii in the pH dependency evaluation in Example 10.

FIG. 20 is a graph showing the relative activity of FADGDH derived from Th. crustaceus in the pH dependency evaluation in Example 10.

FIG. 21 is a graph showing the residual activity percentage of FADGDH derived from Ta. emersonii in the temperature stability evaluation of Example 10.

FIG. 22 is a graph showing the residual activity percentage of FADGDH derived from Th. crustaceus in the temperature stability evaluation of Example 10.

FIG. 23 is a graph showing the relative activity of FADGDH derived from Ta. emersonii in the temperature dependency evaluation of Example 10.

FIG. 24 is a graph showing the relative activity of FADGDH derived from Th. crustaceus in the temperature dependency evaluation of Example 10.

FIG. 25 is a graph showing the current value regarding FADGDH derived from Ta. emersonii (glycosylated) in the responsiveness evaluation in the glucose sensor chip in Example 10.

FIG. 26 is a graph showing the current value regarding FADGDH derived from Ta. emersonii (non-glycosylated) in the responsiveness evaluation in the glucose sensor chip in Example 10.

FIG. 27 is a graph showing the current value regarding FADGDH derived from Th. crustaceus (glycosylated) in the responsiveness evaluation in the glucose sensor chip in Example 10.

FIG. 28 is a graph showing the current value regarding FADGDH derived from Th. crustaceus (non-glycosylated) in the responsiveness evaluation in the glucose sensor chip in Example 10.

### DESCRIPTION OF EMBODIMENTS

### Embodiment 1

The invention of the present embodiment is a protein derived from a thermophilic filamentous fungus, and is a protein (enzyme) having FADGDH activity.

Heretofore, a protein having FADGDH activity derived from a thermophilic filamentous fungus has not been known, and screening for a protein having FADGDH activity derived from a thermophilic filamentous fungus has not been made as a measure for improving the thermal stability of FADGDH. Also, it is actually difficult to screen for a protein having FADGDH activity by analyzing proteins produced by thermophilic filamentous fungi because the production amount of FADGDH is very small.

The present inventors examined a genomic DNA of thermophilic filamentous fungi by a unique technique, and found that some thermophilic filamentous fungi produce a protein having FADGDH activity. To be more specific, focusing on a common part of primary structure (amino acid sequence) of FADGDH derived from normal temperature filamentous fungi (Aspergillus filamentous fungi) that are known to produce FADGDH, degenerate PCR (polymerase chain reaction) was conducted by using a genomic DNA of a thermophilic filamentous fungus as a template and a primer corresponding to a base sequence encoding a specific amino acid sequence in the common part, and the resultant PCR product was analyzed to determine whether the thermophilic filamentous fungus produces a protein having FADGDH activity.

The specific amino acid sequence in the common part of amino acid sequences of FADGDH derived from normal temperature filamentous fungi is preferably YDYIVVGGGTSGL, QVLRAGKALGGTSTINGMAYTRAEDVQID, RSNFHPVGTAAMM, or NVRVVDASVLPFQVCGHLVSTLYAVAERA.

As the primer for degenerate PCR, it is preferred to use a primer including a base sequence encoding at least part of these amino acid sequences or a base sequence that is complementary to the same.

Examples of the base sequence of a sense primer among these preferred primers include:

| (SEQ ID NO: 5) | |
|---|---|
| (A) | 5′-TAYGAYTAYATCGTTYTKGGAGGCGG-3′, |
| (SEQ ID NO: 6) | |
| and | |
| (B) 5′-CAAGTKCTNCGTGCRGGRAAGGCCCTTGG-3′, | |
| (SEQ ID NO: 7) | |
| (C) | 5′-ACSCGCGCMGAGGATGTCCAGAT-3′. |

Examples of the base sequence of an antisense primer include:

(SEQ ID NO: 8)

| | |
|---|---|
| (D) | 5′-CATCATGGCAGCMGTKCCGACGGGRTGGAAGTT-3′, |
| and | |

(SEQ ID NO: 9)

| | |
|---|---|
| (E) | 5′-GTGCTMACCAARTGGCCGCARACCTGGAA-3′. |

Therefore, concrete examples of the protein of the present embodiment include proteins that contain an amino acid sequence encoded by at least one base sequence selected from the group consisting of the base sequences of SEQ ID NOs: 5 to 7 and the base sequences that are complementary to the base sequences of SEQ ID NOs: 8 and 9, and have flavin adenine dinucleotide-dependent glucose dehydrogenase activity.

The thermophilic filamentous fungus is not particularly limited as far as it has FADGDH activity and produces a protein having excellent thermal stability, and the one having an optimum growth temperature of 35° C. or higher or a growth limit temperature of 50° C. or higher is preferred. A more preferred thermophilic filamentous fungus is Talaromyces emersonii (Ta. emersonii) or Thermoascus crustaceus (Th. crustaseus).

Preferably, the protein (enzyme) of the present invention is glycosylated. This is because glycosylated proteins generally tend to be superior in thermal stability or the like to glycosylated proteins. However, the recombinant protein having FADGDH activity of the present invention unexpectedly tends to have relatively excellent thermal stability compared with the conventional case, not only in the case where a sugar chain is added to the surface (glycosylated) (for example, FADGDH produced by recombinant yeast or a wild-type strain of thermophilic filamentous fungus) but also in the case where a sugar chain is not added to the surface (non-glycosylated) (for example, FADGDH produced by recombinant E. coli). Therefore, non-glycosylated recombinant FADGDH may be produced from recombinant E. coli as far as practically acceptable thermal stability is obtainable, and this case is very advantageous in terms of production efficiency, and is capable of greatly reducing the production cost. Thermal stability is determined by measuring FADGDH activity of enzyme after a heat treatment at a predetermined temperature for a predetermine time, and calculating the percentage of the measurement to the FADGDH activity value before the heat treatment. FADGDH activity can be measured in various publicly known methods.

### Embodiment 2

The invention of the present embodiment is a protein of either one of the following (a) to (d):

(a) a protein having an amino acid sequence represented by SEQ ID NO: 1 or 2,

(b) a protein having an amino acid sequence that is the amino acid sequence represented by SEQ ID NO: 1 or 2 from which a signal peptide is removed,

(c) a protein having an amino acid sequence that is the amino acid sequence of the protein of the above (a) or (b) from/to which one or several amino acid residues are deleted, substituted, added or inserted, and

(d) a protein having a homology of more than or equal to 70% with the amino acid sequence of the protein of any one of the above (a) to (c), and having flavin adenine dinucleotide-dependent glucose dehydrogenase activity.

SEQ ID NO: 1 is an “amino acid sequence of FADGDH derived from wild-type Talaromyces emersonii” and contains a signal peptide. SEQ ID NO: 2 is an “amino acid sequence of FADGDH derived from wild-type Thermoascus crustaceus” and contains a signal peptide. Both Talaromyces emersonii and Thermoascus crustaceus are thermophilic filamentous fungi.

A signal peptide is an essential structure for transporting a protein that is biosynthesized in a cell to an appropriate site in the biosynthesis process of a protein molecule by a filamentous fungus or the like. The part of the signal peptide is once synthesized according to the information of a gene encoding an “amino acid sequence containing a signal peptide” like SEQ ID NO: 1, but is finally removed after completion of its role.

Also the protein of the present embodiment is preferably a protein derived from a thermophilic filamentous fungus.

### Embodiment 3

The invention of the present embodiment is a gene encoding the protein of Embodiment 1 or 2. Concrete examples include genes having a DNA of any one of the following (A) to (D):

(A) a DNA having a base sequence according to SEQ ID NO: 3 or 4,

(B) a DNA having a base sequence that is the base sequence of SEQ ID NO: 3 or 4 from which a base sequence encoding a signal peptide is removed,

(C) a DNA containing the base sequence of the DNA of (A) or (B) and an intron, and

(D) a DNA that hybridizes with a DNA having a base sequence complementary to the DNA of any one of (A) to (C) under a stringent condition, and encodes a protein having flavin adenine dinucleotide-dependent glucose dehydrogenase activity.

SEQ ID NO: 3 is a “base sequence of a gene encoding FADGDH derived from wild-type Talaromyces emersonii” and contains a base sequence encoding a signal peptide and a stop codon. SEQ ID NO: 4 is a “base sequence of a gene encoding FADGDH derived from wild-type Thermoascus crustaceus” and contains a base sequence encoding a signal peptide and a stop codon.

The “DNA containing the base sequence of the DNA of (A) or (B) and an intron” refers to a DNA containing the base sequence of the DNA of (A) or (B) as an exon and an intron sequence intervening the exon sequence.

The gene of the present embodiment also includes a gene composed of a DNA that hybridizes with a DNA having a base sequence complementary to the DNA of (A) or (B) under a stringent condition and encodes a protein having GDH activity.

The stringent condition as used herein differs depending on the employed probe and labeling method, and for example, as a condition for hybridization, a condition of “in 5×SSC containing 0.02% SDS at 40 to 70° C.” can be recited. The stringent condition can be determined on the basis of the melting temperature (Tm) of the binding nucleic acid. As a washing condition after hybridization, for example, a condition of “2×SSC, 0.1% SDS, room temperature (25° C.)” can be recited. In the stringent condition, it is preferred that hybridization occurs only when the sequence has a homology of more than or equal to 90%, preferably more than or equal to 95%, and more preferably more than or equal to 97% with the base sequence represented by SEQ ID NO: 3 or 4.

The base sequence of the gene of the present embodiment is not limited to these concrete examples, and may be any base sequence that encodes the protein of Embodiment 1 or 2, and also includes a base sequence in which codon usage is modified to improve the expression of FADGDH.

### Embodiment 4

The invention of the present embodiment is a recombinant vector including the gene of Embodiment 3. The gene of Embodiment 3 is introduced into a host microorganism, for example, in the state that it is connected with a plasmid vector, and the host becomes a transformant that produces FADGDH.

### Embodiment 5

The invention of the present embodiment is a transformant including the gene of Embodiment 3. As the host of the transformant, various hosts including E. cob, yeast, filamentous fungus, animal cell, insect cell and so on can be used depending on the purpose, however, E. coli, or microbial eukaryotes such as yeast and filamentous fungus is preferably used. From the view point of production efficiency, E. coli is preferably used. Examples of E. coli include E. coli JM109, E. coli DH5α, E. coli W3110, and E. coli C600. Also, it is preferred to use eukaryotes as a host for producing a glycosylated protein. Among the eukaryotes, yeast is often employed industrially, and for example, Pichia pastoris (P. pastoris), Saccharomyces cerevisiae, or Schizosaccharomyces pombe can be used.

Concrete examples of the present embodiment include E. coli and yeast transformed with the recombinant vector of Embodiment 4.

### Embodiment 6

The invention of the present embodiment is a method for producing FADGDH derived from a thermophilic filamentous fungus using the gene of Embodiment 3. In the present embodiment, for example, by using the transformant of Embodiment 5, FADGDH can be produced. A concrete exemplary procedure for production is as follows.

A DNA having genetic information of FADGDH derived from a thermophilic filamentous fungus (gene of Embodiment 3) is introduced into a host in the state that it is connected with a plasmid vector. The host is similar to that described in the above Embodiment 5. When a protein having FADGDH activity is produced in E. coli, it is sometimes impossible to obtain sufficient productivity in the state that the signal peptide at N terminus of the gene remains. Therefore, it is preferred to introduce the gene from which the signal peptide (amino acid sequence of a putative signal peptide based on the genomic information) has been deleted into E. coli.

As a method of introducing a recombinant vector into a host, a method of introducing the recombinant DNA in the presence of calcium ions may be employed when the host is a microorganism categorized in E. coli. Also an electroporation method may be used. Also, a commercially available competent cell (for example, Competent high JM109; available from TOYOBO CO., LTD.) may be used. When the host is yeast, a spheroplast method or a lithium acetate method is used. Also an electroporation method or the like may be used. When the host is a filamentous fungus, a protoplast cell or the like is used.

The microorganism that is a transformant obtained in this manner is capable of producing a large amount of FADGDH stably by being cultured in a nutritive culture medium. Regarding the culture form of the host microorganism which is a transformant, the culture condition may be selected in consideration of the nutritional physiological properties of the host, and the culture is often conducted by liquid culture, and advantageously conducted by aerobic culture under stirring from the industrial view point.

As nutrient sources of the culture medium, those ordinarily used in culture of microorganisms are widely used. As the carbon source, any carbon compounds that can be assimilated may be used, and examples of such compounds include glucose, sucrose, lactose, maltose, molasses, and pyruvic acid. As the nitrogen source, any nitrogen compounds that are available may be used, and examples of such compounds include peptone, meat extract, yeast extract, casein hydrolysate, and soybean cake alkali decomposition product. Besides these, phosphates, carbonates, sulfates, salts of magnesium, calcium, potassium, iron, manganese, zinc and so on, specific amino acids, specific vitamins and so on are used as is necessary.

The temperature of the culture medium can be appropriately changed as far as the bacterium grows and produces FADGDH, and is preferably about 20 to 42° C. in the case of E. coli, and about 20 to 35° C. in the case of yeast. The culture time differs depending on the culture conditions, and the culture may be appropriately halted when the FADGDH reaches the maximum yield. The culture time is typically about 6 to 72 hours. The pH of the culture medium may be appropriately varied as far as the bacterium grows and produces FADGDH, and is particularly preferably about pH 5.0 to 9.0.

The culture liquid containing bacterial cells that produce FADGDH may be directly collected from the culture and used, however, generally, when FADGDH exists in the culture liquid, it is used after the solution containing a protein is separated from the microbial cells by filtration, centrifugal separation or the like. On the other hand, when FADGDH exists in bacterial cells, the bacterial cells are collected from the obtained culture by a technique such as filtration or centrifugal separation, and then the bacterial cells are broken by a mechanical means or an enzymatic means such as lysozyme, and a chelator such as EDTA and a surfactant are added as is necessary to solubilize FADGDH, and thus FADGDH is separated and collected as an aqueous solution.

As a method for collecting FADGDH from the FADGDH-containing solution obtained in this manner, for example, concentration under reduced pressure, membrane concentration, salting-out using ammonium sulfate or sodium sulfate, or fractional precipitation using a hydrophilic organic solvent (e.g. methanol, ethanol, or acetone) can be recited. Also, heating and an isoelectric point treatment are effective purification means. Also, FADGDH may be purified by gel filtration using an adsorbent or a gel filtration agent, adsorption chromatography, ion exchange chromatography, or affinity chromatography.

The production method of FADGDH derived from a thermophilic filamentous fungus is not limited to these methods, and for example, FADGDH derived from a thermophilic filamentous fungus may be produced by a cell-free protein translation system using the gene of Embodiment 3.

### Embodiment 7

The invention of the present embodiment is a glucose sensor using a glucose concentration measuring reagent containing the protein of Embodiment 1 or 2.

The glucose concentration measuring reagent contains FADGDH produced by the method of Embodiment 6 at least in an amount that is sufficient for single measurement. The glucose concentration measuring reagent may contain, for example, a buffer required for assay, a mediator, and a glucose standard solution for preparing a calibration curve besides the FADGDH. The form of the glucose concentration measuring reagent is not particularly limited, and it can be provided in various forms (for example, a lyophilized reagent or a solution in an appropriate preservation container) suited for a glucose sensor.

The electrode for use in a glucose sensor is not particularly limited, and a carbon electrode, a gold electrode, a platinum electrode and so on can be used. For example, on this electrode, the protein of the present invention (enzyme: FADGDH) is immobilized.

As the immobilization method, a method of using a cross-linking reagent, a method of encapsulating in a polymer matrix, a method of covering with a dialysis membrane, a photo-crosslinkable polymer, a conductive polymer, and an oxidation-reduction polymer are recited, or a method of immobilizing in a polymer or fixing on an electrode by adsorption together with an electron mediator as typified by ferrocene or its derivative, or a method combining these methods may be used. Typically used is a method of immobilizing the protein (FADGDH) of the present invention on a carbon electrode by using glutaraldehyde, and then cross-linking the glutaraldehyde by treatment with a reagent having an amine group.

### Embodiment 8

The invention of the present embodiment is a glucose concentration measuring method using the protein of Embodiment 1 or 2. Measurement of glucose concentration can be conducted, for example, in the following manner.

A constant-temperature cell is charged with a buffer, and kept at a constant temperature. As a mediator, potassium ferricyanide, phenazinemethosulfate or the like can be used. As a working electrode, an electrode on which the modified FADGDH of the present invention is immobilized is used, and a counter electrode (e.g., platinum electrode) and a reference electrode (e.g., Ag/AgCl electrode) are used. A constant voltage is applied to the carbon electrode, and after the current becomes steady, a sample containing glucose is added and an increase in the current is measured. According to the calibration curve prepared by using a glucose solution of standard concentration, the glucose concentration in the sample can be calculated.

### Embodiment 9

The invention of the present embodiment is a biofuel cell using the protein (FADGDH) of Embodiment 1 or 2. FADGDH catalyzes dehydrogenation of glucose, and electrons arising by this reaction are supplied as electric power.

### EXAMPLES

Hereinafter, the present invention will be described more specifically by way of examples, however, the present invention is not limited to these examples.

### Example 1

For 17 species of thermophilic filamentous fungi shown in Table 1 purchased from National Institute of Technology and Evaluation, Biotechnology Center, Biological Resource Center (NBRC), screening for a FADGDH gene derived from a thermophilic filamentous fungus was conducted.

TABLE 1

| Thermophilic filamentous fungus | NBRC number |
|---|---|
| Chaetomium thermophilum var. coprophilum | 9679 |
| Chaetomium thermophilum var. coprophilum | 30072 |
| Chaetomium thermophilum var. dissitum | 31807 |
| Malbranchea cinnamomea | 8834 |
| Malbranchea cinnamomea | 9739 |
| Melanocarpus albomyces | 9683 |
| Melanocarpus albomyces | 30079 |
| Myceliophthora hinnulea | 31379 |
| Myceliophthora thermophila | 9219 |
| Myceliophthora thermophila | 9746 |
| Paecilomyces variotii | 7563 |
| Paecilomyces variotii | 33284 |
| Rhizomucor miehei | 9740 |
| Rhizomucor miehei | 9741 |
| Rhizomucor pusillus | 4578 |
| Rhizomucor pusillus | 4579 |
| Talaromyces byssochlamydoides | 31900 |
| Talaromyces byssochlamydoides | 32236 |
| Talaromyces emersonii | 31232 |
| Talaromyces emersonii | 9734 |
| Talaromyces thermophilus | 30541 |
| Talaromyces thermophilus | 31798 |
| Thermoascus aurantiacus | 6766 |
| Thermoascus aurantiacus | 9748 |
| Thermoascus crustaceus | 9129 |
| Thermoascus crustaceus | 9816 |
| Thermoascus thermophilus | 9643 |
| Thermomyces lanuginosus | 9738 |
| Thermomyces lanuginosus | 9863 |
| Thielavia minuta | 31063 |
| Thielavia terrestris | 9121 |
| Thielavia terrestris | 9732 |

Each purchased thermophilic filamentous fungus was aerobically cultured in a potato dextrose culture medium (Difco Laboratories) or a malt extract culture medium (2% malt extract, 2% glucose, 0.1% pepton). From the cultured thermophilic filamentous fungus, a genomic DNA was extracted by using Wizard Genomic DNA Purification Kit (Promega).

Focusing on the previously reported common part of primary structure (amino acid sequence) of FADGDH derived from Aspergillus filamentous fungi (Aspergillus oryzae, Aspergillus terreus), degenerate PCR (polymerase chain reaction) was conducted by using the later-described primers specially designed by the present inventors, and a genomic DNA of thermophilic filamentous fungus as a template.

As a sense primer for degenerate PCR, any one of the following primers was used:

| | |
|---|---|
| primer A | (5′-TAYGAYTAYATCGTTYTYGGAGGCGG-3′), |
| primer B | (5′-CAAGTKCTNCGTGCRGGRAAGGCCCTTGG-3′), |
| and | |
| primer C | (5′-ACSCGCGCMGAGGATGICCAGAT-3′) |

As an antisense primer, either one of the following primers was used:

| | |
|---|---|
| primer D | (5′-CATCATGGCAGCMGTKCCGACGGGRTGGAAGTT-3′), |
| and | |
| primer E | (5′-GTGCTMACCAARTGGCCGCARACCTGGAA-3′). |

The above primers A to E contain mix bases (K, N, M, R, S, and Y) in their sequences. It means that each of primers A to E is a mixture of plural kinds of primers having different base sequences in the positions of the mix bases.

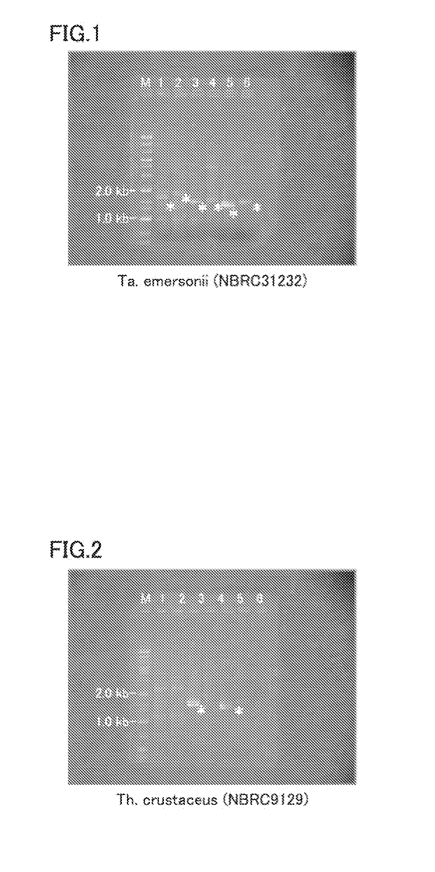

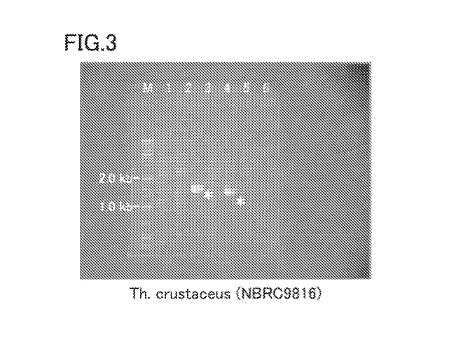

Degenerate PCR was conducted in six different ways based on the combination of the three kinds of sense primers and the two kinds of antisense primers using a genomic DNA obtained from each of 17 species of thermophilic filamentous fungi as a template. The obtained PCR product was subjected to agarose gel electrophoresis (1.2% agarose gel), and the gel after migration was stained with ethidium bromide to confirm the presence of the PCR product. As a result, a DNA band of about 1.8 kb was detected in the PCR products obtained by using genomes of the three thermophilic filamentous fungi: Ta. emersonii (NBRC31232), Th. crustaceus (NBRC9129), and Th. crustaceus (NBRC9816) as templates (the marks * in FIG. 1 to FIG. 3). In FIGS. 1 to 3, lane M is a marker, lane 1 is the case using a combination of primers A and D, lane 2 is the case using a combination of primers A and E, lane 3 is the case using a combination of primers B and D, lane 4 is the case using a combination of primers B and E, lane 5 is the case using a combination of primers C and D, and lane 6 is the case using a combination of primers C and E.

The DNA fragment amplified by degenerate PCR was connected with pCR-Blunt-II-TOPO (Life Technologies), and then subcloned into E. coli DH5α (TAKARA SHUZO CO., LTD.). The base sequence of the inserted DNA fragment was analyzed, and amplification of a gene fragment having high homology with a previously reported FADGDH gene derived from Aspergillus filamentous fungus was confirmed. This suggests the presence of a FADGDH gene in genomes of Ta. emersonii and Th. crustaceus. NBRC9129 and NBRC9816 of Th. crustaceus contained the same base sequence of a FADGDH gene.

When the screening was conducted by using several primers other than the aforementioned primers A to E, amplification of a gene fragment having high homology with a FADGDH gene derived from Aspergillus filamentous fungus was not observed.

### Example 2

In the present example, FADGDH derived from Ta. emersonii was produced by using E. coli. Hereinafter, the details will be described.

(1) Cloning of FADGDH Gene from Ta. emersonii (NBRC31232)

Before cloning, a genomic DNA was subjected to a Southern hybridization analysis using a partial fragment of Ta. emersonii-derived FADGDH gene as a probe. Ta. emersonii genomic DNA was digested with various restriction enzymes (New England BioLabs), and subjected to agarose gel electrophoresis. After migration, the DNA fragment contained in the gel was transcribed to nylon membrane (Hybond-N+; GE Healthcare) by a capillary method using 20×SSC (3 M NaCl, 0.3 M sodium citrate, pH 7.0). The nylon membrane after transcription and digoxigenin-labeled probe DNA (DIG DNA Labeling and Detection Kit; Roche) were mixed together, and allowed to hybridize at 65° C. The DNA fragment hybridizing with the probe was detected by using alkaline phosphatase-labeled anti-DIG antibody (DIG DNA Labeling and detection Kit).

As a result, a DNA band that hybridizes with the probe was detected at the position of about 4.0 kb in the lane of EcoRI. Since the open reading frame (ORF) of a previously reported FADGDH gene of Aspergillus filamentous fungus is about 1.8 kb, it was considered that this EcoRI fragment of about 4.0 kB hybridizing with the probe included the whole region of Ta. emersonii-derived FADGDH gene.

Next, cloning of a 5′-flanking region including the initiation codon neighboring region of Ta. emersonii-derived FADGDH gene and a 3′-flanking region including the stop codon neighboring region was attempted by means of Inverse PCR. A genomic DNA of Ta. emersonii cut with EcoRI was applied to a preparative agarose gel, and a DNA fragment of about 4.0 kb was collected. This DNA fragment was cyclized by T4 ligase (TAKARA SHUZO CO., LTD.) and provided as a template for Inverse PCR.

Inverse PCR was conducted by using primer 31232GSP3 (5′-GTTGTTGGGCTGCATCTCGATGAAGCC-3′) and primer 31232GSP5 (5′-CCATCTGTGACGGACCTCTTTGGCAACCGC-3′) that were designed based on the internal sequence of Ta. emersonii-derived FADGDH gene, and a single DNA fragment was detected at the position of about 3.0 kb.

This DNA fragment was subcloned, and subjected to a base sequence analysis. The analysis revealed the success in amplifying the 5′-flanking region and the 3′-flanking region of Ta. emersonii-derived FADGDH gene.

Next, PCR using primers 31232GSP9 (5% GCATGCCGAGGACTACTTGTATTCCGGGAT-3′) and 31232GSP10 (5′-GATATCTGCATTTCGGGCATTCCTATCTCT-3′) designed based on the sequences of 5′-flanking region and 3′-flanking region, and a genomic DNA derived from Ta. emersonii as a template was conducted. As a result, amplification of a single DNA fragment of about 2.8 kb was confirmed, revealing that the DNA fragment included the whole region of Ta. emersonii-derived FADGDH gene.

(2) Analysis of Base Sequence of Ta. emersonii-Derived FADGDH Gene

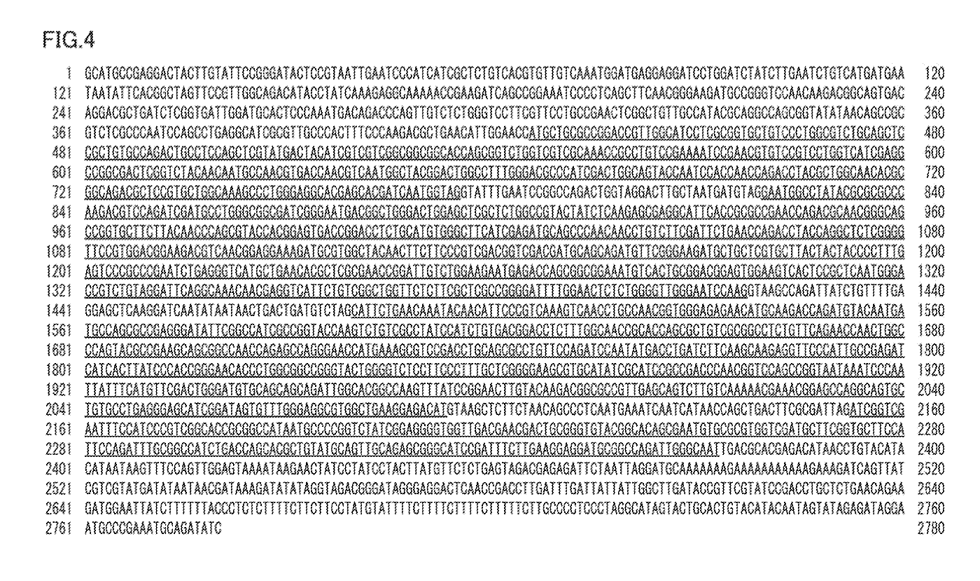

The base sequence of the DNA fragment including the whole region of Ta. emersonii-derived FADGDH gene was determined by a primer walking method. The base sequence of the DNA fragment including the whole region of Ta. emersonii-derived FADGDH gene is shown in FIG. 4.

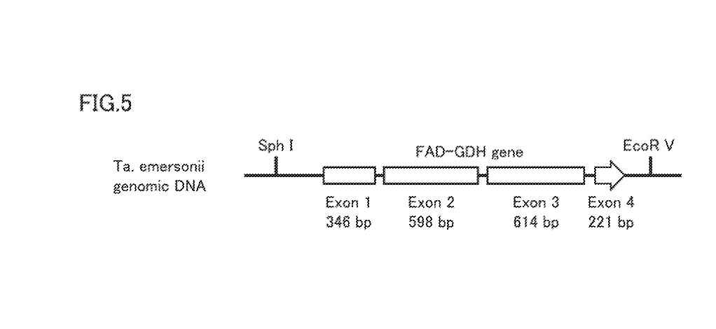

By predicting the splicing site on the basis of the GT-AG rule, exons and introns in this base sequence were identified. Ta. emersonii-derived FADGDH gene included ORF of 1779 bp (encoding 593 amino acids) consisting of four exons (exon 1; 346 bp, exon 2; 598 bp, exon 3; 614 bp, exon 4; 221 bp) (see the underlined part in FIG. 4, and FIG. 5).

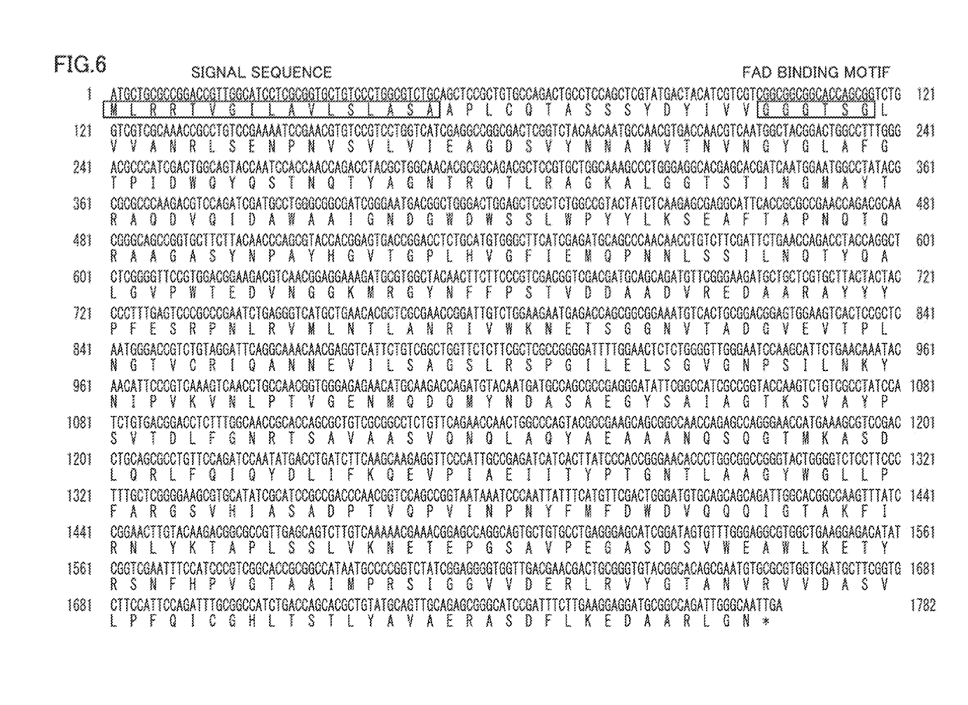

FIG. 6 shows the base sequence of ORF (FADGDH coding region) of Ta. emersonii-derived FADGDH gene identified in this manner, and the amino acid sequence analogized from the base sequence.

For the amino acid sequence shown in FIG. 6, presence or absence of a signal peptide, and a cleavage site of the signal peptide were predicted by the signal peptide predicting server SignalP 4.0 (http://www.cbs.dtu.dk/services/SignalP/). As a result, the sequence from methionine (Met1) coming from the initiation codon to the seventeenth alanine (Ala17) was identified as a signal peptide. In the amino acid sequence shown in FIG. 6, a FAD binding motif (Gly-Gly-Gly-Thr-Ser-Gly) that is important for FAD binding was completely conserved.

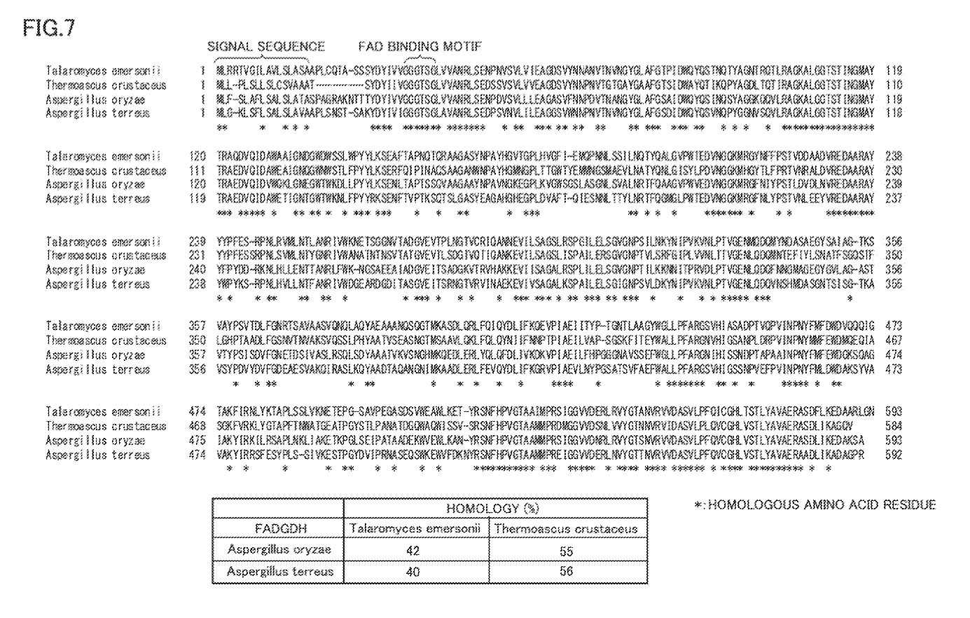

Further, the amino acid sequence of FADGDH derived from Ta. emersonii (FIG. 6) was compared with FADGDH derived from Aspergillus filamentous fungi, and it had a homology of 42% with FADGDH derived from Aspergillus oryzae and a homology of 40% with FADGDH derived from Aspergillus terreus (FIG. 7).

(3) Preparation of Ta. emersonii-Derived FADGDH Gene Expression Type Plasmid

The coding region of Ta. emersonii-derived FADGDH gene was synthesized by Overlap extension PCR.

First, each exon was amplified by PCR. For PCR amplification of exon 1, 31232GSP11 (5′-ACCATGCTGCGCCGGACCGTTGGCATCCTC-3′) and 31232E1A (5′-GGCCATTCCATTGATCGTGCTCGTGCCTCC-3′) were used as primers. For PCR amplification of exon 2, 31232E2S (5′-ATCAATGGAATGGCCTATACGCGCGCCCAA-3′) and 31232E2A (5′-CAGAATGCTTGGATTCCCAACCCCAGAGAG-3′) were used as primers. For PCR amplification of exon 3, 31232E3S (5-AATCCAAGCATTCTGAACAAATACAACATT-3) and 31232E3A (5′-CGACCGATATGTCTCCTTCAGCCACGCCTC-3) were used as primers. For PCR amplification of exon 4, 31232E4S (5′-GAGACATATCGGTCGAATTTCCATCCCGTC-3′) and 31232GSP12 (5′-GCGTCAATTGCCCAATCTGGCCGCATCCTC-3′) were used as primers.

The amplified exon 1 and exon 2 were connected by Overlap extension PCR, and also exon 3 and exon 4 were connected by Overlap extension PCR. These DNA fragments synthesized by Overlap extension PCR were again connected by Overlap extension PCR to synthesize the coding region of Ta. emersonii-derived FADGDH gene.

Next, PCR was conducted using the coding region of Ta. emersonii-derived FADGDH gene as a template, and primers 31232Esc2 (5′-AAACATATGGCTCCGCTGTGCCAGACTGCC-3′) and 31232Esc4 (5′-TTTAAGCTTTTAGTGGTGGTGGTGGTGGTGATTGCCCAATCTGGCCGCATC-3′) for preparation of expression type plasmid to amplify the gene encoding matured FADGDH. On the C-terminal side of the amplified gene that encodes Ta. emersonii matured FADGDH, the codon (CAC) for histidine for fusion with a histidine tag is added.

The amplified DNA fragment was cut with NdeI and HindIII, and then connected in the equivalent restriction enzyme site of expression vector pET-21b (+) (Novagen) for E. coli to prepare Ta. emersonii-derived FADGDH gene expression type plasmid.

(4) Expression of Ta. emersonii-Derived FADGDH Gene in E. coli, and Purification of Recombinant Enzyme

Ta. emersonii-derived FADGDH gene expression type plasmid was introduced into E. coli BLR (DE3) strain (Novagen). BLR (DE3) having the expression type plasmid was cultured overnight in an LB culture medium (containing 100 μg/mL ampicillin) (pre-culture). In an LB culture medium for main culture (containing 100 μg/mL ampicillin), the pre-culture medium was inoculated at 1%, and was aerobically cultured at 37° C. for about 4 hours. Then, isopropyl-β-D-thiogalactopyranoside (IPTG) was added at a final concentration of 0.1%, and the culture medium was continuously cultured at 25° C. for about 20 hours.

E. coli after the culture was collected, and suspended in a PBS buffer (140 mM NaCl, 2.7 mM KCl, 8.1 mM disodium hydrogenphosphate dodecahydrate, 1.5 mM potassium dihydrogenphosphate, pH 7.4) to wash the bacterial cells. The bacterial cells were again collected by centrifugal separation, and suspended in a PBS buffer. The bacterial cells suspended in the PBS buffer were crushed ultrasonically. The disrupted cell suspension after the ultrasonic crushing was centrifuged (8000 g, 10 minutes, 4° C.) to separate it into a supernatant (whole cell fraction) and a precipitate (uncrushed bacterial cell). The whole cell fraction was further centrifuged (12000 g, 20 minutes, 4° C.), and was fractioned into a soluble fraction and an insoluble fraction. The soluble fraction was applied to Ni-NTA Agarose (QIAGEN) column (diameter 0.7 cm, height 1.0 cm) equilibrated with a PBS buffer. After washing the column with a PBS buffer, the adsorbed protein was eluted with 20 mM Hepes-NaOH buffer (pH 7.5) containing 100 mM imidazole.

The eluted fraction was applied to Q Sepharose Fast Flow (GE Healthcare) column (diameter 0.7 cm, height 2.5 cm) equilibrated with 20 mM Hepes-NaOH buffer (pH 7.5). After washing the column with 20 mM Hepes-NaOH (pH 7.5), the adsorbed protein was eluted with 20 mM Hepes-NaOH buffer (pH 7.5) containing 100 mM NaCl. The eluted fraction was subjected to demineralization by using Amicon Ultra-15 MWCO10 (Millipore), and applied to Resource Q 1 mL (GE Healthcare) equilibrated with 20 mM Hepes-NaOH buffer (pH 7.5). The adsorbed protein was eluted by 0 to 1.0 M NaCl linear concentration gradient (20 column volume). The eluted fraction was subjected to demineralization using Amicon Ultra-4 MWCO10 (Millipore) to give a Ta. emersonii-derived FADGDH purified sample.

We also prepared Ta. emersonii-derived FADGDH gene expression type plasmid to which a gene encoding a signal peptide is added, and attempted to let it be expressed in E. coli, however, expression was not observed.

### Example 3

In the present example, Th. crustaceus (NBRC9129)-derived FADGDH was produced by using E. coli. Hereinafter, the details will be described.

(1) Cloning of FADGDH Gene from Th. crustaceus (NBRC9129)

Before cloning, a genomic DNA was subjected to a Southern hybridization analysis using a partial fragment of Th. crustaceus-derived FADGDH gene. Th. crustaceus genomic DNA was digested with various restriction enzymes, and subjected to agarose gel electrophoresis. After migration, the DNA fragment contained in the gel was transcribed to nylon membrane by a capillary method using 20×SSC. The nylon membrane after transcription and digoxigenin-labeled probe DNA were mixed together, and allowed to hybridize at 65° C. The DNA fragment hybridizing with the probe was detected by using alkaline phosphatase-labeled anti-DIG antibody.

As a result, a DNA band that hybridizes with the probe was detected at the position of about 5.0 kb in the lane of HindIII. Since the ORF of a previously reported FADGDH gene of Aspergillus filamentous fungus is about 1.8 kb, it was considered that this HindIII fragment of about 5.0 kb hybridizing with the probe included the whole region of Th. crustaceus-derived FADGDH gene.

Next, cloning of a 5′-flanking region including the initiation codon neighboring region of Th. crustaceus-derived FADGDH gene and a 3′-flanking region including the stop codon neighboring region was attempted by means of Inverse PCR.

A genomic DNA of Th. crustaceus cut with HindIII was applied to a preparative agarose gel, and a DNA fragment of about 5.0 kb was collected. This DNA fragment was cyclized by T4 ligase and provided as a template for Inverse PCR. Inverse PCR was conducted by using primers 9129GSP3 (5′-GCTGCCGTTCATCATCTCGTAGGTCCAGCC-3′), and 9129GSP5 (5′-GGCAGCAACGTGACAAACGTGGCCAAG-3′) that were designed based on the internal sequence of Th. crustaceus-derived FADGDH gene, and a single DNA fragment was detected at the position of about 3.5 kb. This DNA fragment was subcloned, and subjected to a base sequence analysis, and the 5′-flanking region and the 3′-flanking region of Th. crustaceus-derived FADGDH gene were successfully amplified.

Next, PCR using primers 91229GSP9 (5′-CTCGAGCAATTTAATTTATGCTTCGACGGT-3′) and 9129GSP10 (5′-AGATCTCCATTCCCGCTGGACCTGGACGGT-3′) designed based on the sequences of 5′- and 3′-flanking regions, and a genomic DNA derived from Th. crustaceus as a template was conducted.

As a result, amplification of an electrophoretically single DNA fragment of about 2.6 kb was confirmed, revealing that the DNA fragment included the whole region of Th. crustaceus-derived FADGDH gene.

(2) Analysis of Base Sequence of Th. crustaceus-Derived FADGDH Gene

The base sequence of the DNA fragment including the whole region of Th, crustaceus-derived FADGDH gene was determined by a primer walking method. The base sequence of the DNA fragment including the whole region of Th. crustaceus-derived FADGDH gene is shown in FIG. 8.

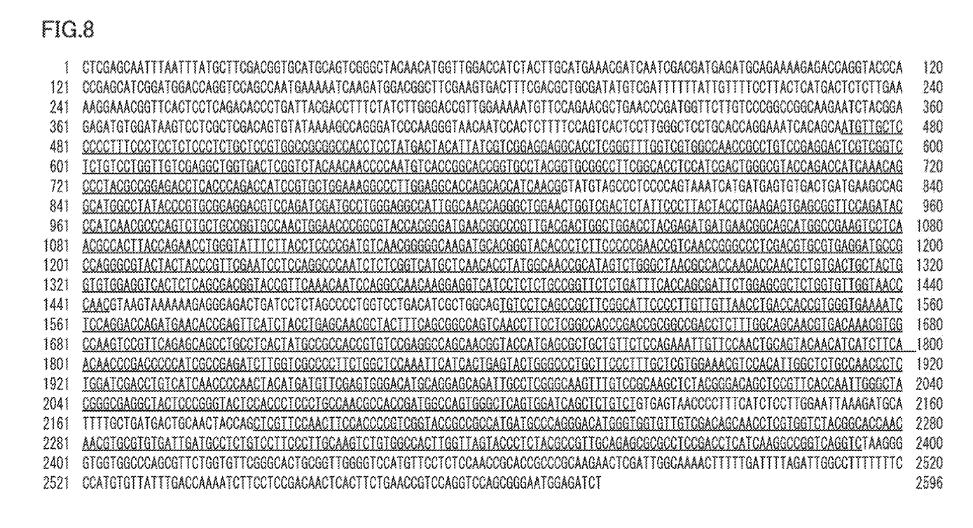

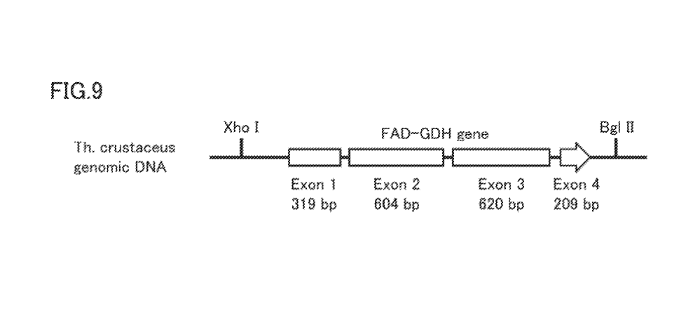

By predicting the splicing site on the basis of the GT-AG rule, exons and introns in this base sequence were identified. Th. crustaceus-derived FADGDH gene included ORF of 1752 bp (encoding 594 amino acids) consisting of four exons (exon 1; 319 bp, exon 2; 604 bp, exon 3; 620 bp, exon 4; 209 bp) (see the underlined part in FIG. 8, and FIG. 9).

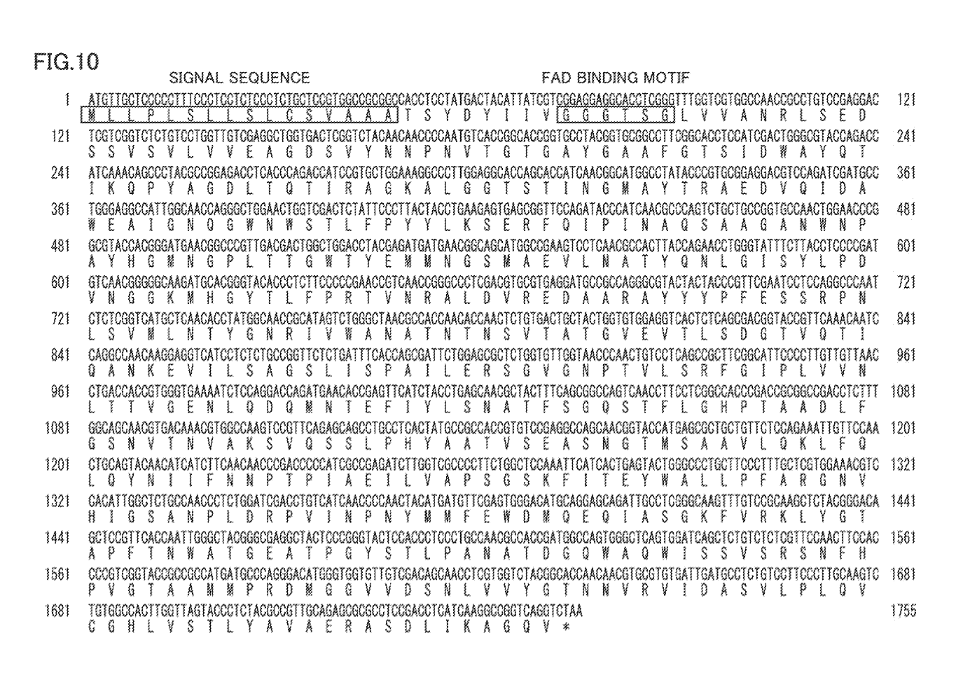

FIG. 10 shows the base sequence of ORF (FADGDH coding region) of Th. crustaceus-derived FADGDH gene identified in this manner, and the amino acid sequence analogized from the base sequence.

For the amino acid sequence shown in FIG. 10, presence or absence of a signal peptide, and a cleavage site of the signal peptide were predicted by the signal peptide predicting server SignalP 4.0. As a result, the sequence from methionine (Met1) coming from the initiation codon to the sixteenth alanine (Ala16) was identified as a signal peptide. In the amino acid sequence shown in FIG. 10, a FAD binding motif (Gly-Gly-Gly-Thr-Ser-Gly) that is important for FAD binding was completely conserved.

Further, the amino acid sequence of FADGDH derived from Th. crustaceus (FIG. 10) was compared with FADGDH derived from Aspergillus filamentous fungi, and it had a homology of 55% with FADGDH derived from Aspergillus oryzae and a homology of 56% with FADGDH derived from Aspergillus terreus (FIG. 7). The amino acid sequence of FADGDH derived from Ta. emersonii (FIG. 6) and the amino acid sequence of FADGDH derived from Th. crustaceus (FIG. 10) had a homology of 62%.

(3) Preparation of Th. crustaceus-Derived FADGDH Gene Expression Type Plasmid

The coding region of Th. crustaceus-derived FADGDH gene was synthesized by Overlap extension PCR.

First, each exon was amplified by PCR. For PCR amplification of exon 1, 9129GSP11 (5′-GCAATGTTGCTCCCCCTTTCCCTCCTCTCC-3′) and 9129E1A (5′-GGCCATGCCGTTGATGGTGCTGGTGCCTCC-3′) were used as primers. For PCR amplification of exon 2, 9129E2S (5′-ATCAACGGCATGGCCTATACCCGTGCGGAG-3′) and 9129E2A (5′-GAGGACAGTTGGGTTACCAACACCAGAGCG-3′) were used as primers. For PCR amplification of exon 3, 9129E3S (5-AACCCAACTGTCCTCAGCCGCTTCGGCATT-3) and 9129E3A (5′-GGAACGAGAGACAGAGCTGATCCACTGAGC-3) were used as primers. For PCR amplification of exon 4, 9129E4S (5′-TCTGTCTCTCGTTCCAACTTCCACCCCGTC-3′) and 9129GSP12 (5′-CCCTTAGACCTGACCGGCCTTGATGAGGTC-3′) were used as primers.

The amplified exon 1 and exon 2 were connected by Overlap extension PCR, and also exon 3 and exon 4 were connected by Overlap extension PCR. These DNA fragments synthesized by Overlap extension PCR were again connected by Overlap extension PCR to synthesize the coding region of Th. crustaceus-derived FADGDH gene.

Next, PCR was conducted using the coding region of Th. crustaceus-derived FADGDH gene as a template, and primers 9129Esc2 (5′-AAACATATGACCTCCTATGACTACATTATC-3′) and 9129Esc4 (5′-TTTAAGCTTTTAGTGGTGGTGGTGGTGGTGGACCTGACCGGCCTTGATGAG-3′) for preparation of expression type plasmid to amplify the gene encoding matured FADGDH. On the C-terminal side of the amplified gene that encodes Th. crustaceus matured FADGDH, the codon (CAC) for histidine for fusion with a histidine tag is added.

The amplified DNA fragment was cut with NdeI and HindIII, and then connected in the equivalent restriction enzyme site of expression vector pET-21b (+) (Novagen) for E. coli to prepare Th. crustaceus-derived FADGDH gene expression type plasmid.

(4) Expression of Th. crustaceus-Derived FADGDH Gene in E. coli, and Purification of Recombinant Enzyme

Th. crustaceus-derived FADGDH gene expression type plasmid was introduced into E. coli BLR (DE3) strain (Novagen). BLR (DE3) having the expression type plasmid was cultured overnight in an LB culture medium (containing 100 μg/mL ampicillin) (pre-culture). In an LB culture medium for main culture (containing 100 μg/mL ampicillin), the pre-culture medium was inoculated at 1%, and was aerobically cultured at 37° C. for about 4 hours. Then, isopropyl-β-D-thiogalactopyranoside (IPTG) was added at a final concentration of 0.1%, and the culture medium was continuously cultured at 25° C. for about 20 hours.

E. coli after the culture was collected, and suspended in a PBS buffer to wash the bacterial cells. The bacterial cells were again collected by centrifugal separation, and suspended in a PBS buffer. The bacterial cells suspended in the PBS buffer were crushed ultrasonically. The disrupted cell suspension after the ultrasonic crushing was centrifuged (8000 g, 10 minutes, 4° C.) to separate it into a supernatant (whole cell fraction) and a precipitate (uncrushed bacterial cell). The whole cell fraction was further centrifuged (12000 g, 20 minutes, 4° C.), and was fractioned into a soluble fraction and an insoluble fraction. The soluble fraction was applied to Ni-NTA Agarose (QIAGEN) column (diameter 0.7 cm, height 1.0 cm) equilibrated with a PBS buffer. After washing the column with a PBS buffer, the adsorbed protein was eluted with 20 mM Hepes-NaOH buffer (pH 7.5) containing 100 mM imidazole.

The eluted fraction was applied to Q Sepharose Fast Flow (GE Healthcare) column (diameter 0.7 cm, height 2.5 cm) equilibrated with 20 mM Hepes-NaOH buffer (pH 7.5). After washing the column with 20 mM Hepes-NaOH buffer (pH 7.5), the adsorbed protein was elated with 20 mM Hepes-NaOH buffer (pH 7.5) containing 100 mM NaCl. The eluted fraction was subjected to demineralization by using Amicon Ultra-15 MWCO10 (Millipore), and applied to Resource Q 1 mL (GE Healthcare) equilibrated with 20 mM Hepes-NaOH buffer (pH 7.5). The adsorbed protein was elated by 0 to 1.0 M NaCl linear concentration gradient (20 column volume). The eluted fraction was subjected to demineralization using Amicon Ultra-4 MWCO10 (Millipore) to give a Th. crustaceus-derived FADGDH purified sample.

We also prepared Th. crustaceus-derived FADGDH gene expression type plasmid to which a gene encoding a signal peptide is added, and attempted to let it be expressed in E. coli. Th, crustaceus-derived FADGDH was produced as an insoluble inclusion body in E. coli cells.

### Example 4

### SDS-PAGE

The protein concentration in each FADGDH purified sample obtained in Examples 2 and 3 (solutions containing Ta. emersonii-derived FADGDH and Th. crustaceus-derived FADGDH expressed in E. coli and purified) was measured. The protein concentration was measured by the BCA method using a commercially available protein quantification kit (Pierce BCA Protein Assay Reagent; Thermo Fisher Scientific K.K.). In this kit, bovine serum albumin is used as a standard protein. Based on the obtained measured concentration value, the purified sample containing 5 μg of FADGDH was subjected to SDS-polyacrylamide electrophoresis (SDS-PAGE).

The SDS-PAGE was conducted according to the method of Laemmli. As SDS-polyacrylamide gel, “e-PAGEL 12.5%” (ATTO Corporation) was used. The gel after migration was stained with a CBB (Coomassie Brilliant Blue)-based stain solution “GelCode Blue Safe Protein Stain” (Thermo Fisher Scientific K.K.). As a protein marker, “Protein Ladder (10-250 kDa)” (New England BioLabs) was used.

The result of SDS-PAGE is shown in FIG. 11. As shown in FIG. 11, it was proved that for both of FADGDH derived from Ta. emersonii and FADGDH derived from Th. crustaceus, electrophoretically single purified samples were successfully obtained.

### Example 5

### Measurement of Absorption Spectrum

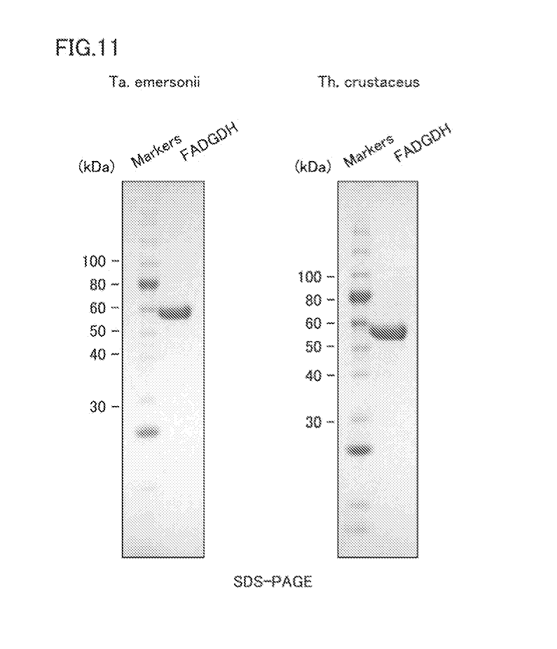

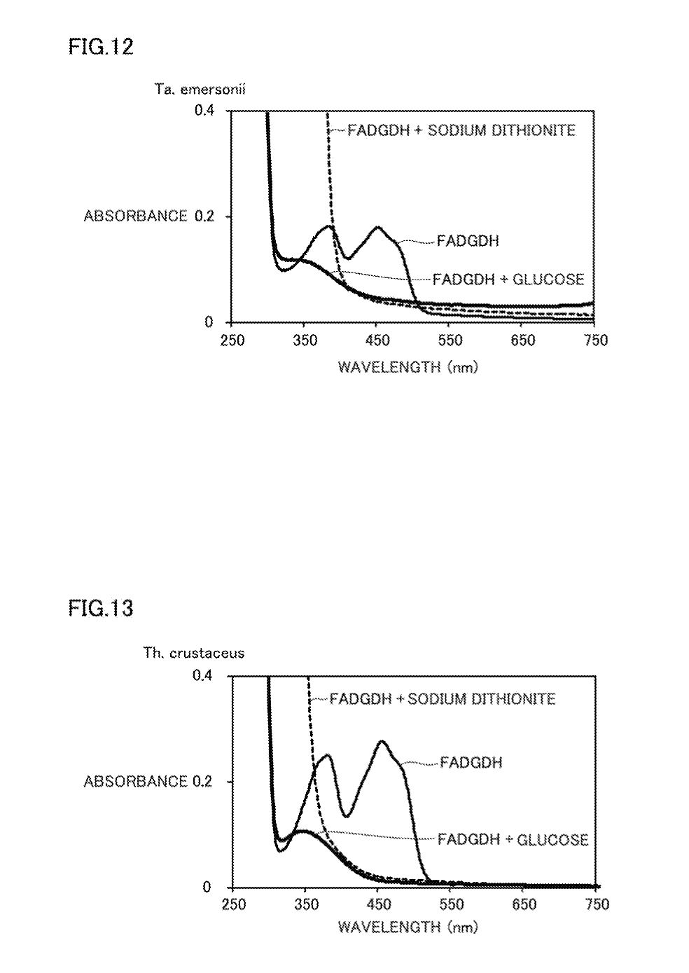

Each FADGDH purified sample obtained in Examples 2 and 3 (solutions containing Ta. emersonii-derived FADGDH and Th. crustaceus-derived FADGDH expressed in E. coli and purified) was diluted based on the measured concentration value in Example 4, and a 500 μg/mL enzyme solution (FADGDH solution) was prepared. An absorption spectrum of the FADGDH solution was measured by using a UV/visible spectrophotometer DU-800 (Beckman Coulter). Also for the solution prepared by adding glucose to the FADGDH solution in a final concentration of 100 mM, and for the solution prepared by adding sodium dithionite powder which is a reducing agent to the FADGDH solution, an absorption spectrum was measured in a similar manner. The obtained absorption spectra are shown in FIG. 12 (FADGDH derived from Ta. emersonii), and FIG. 13 (FADGDH derived from Th. crustaceus).

As shown in FIGS. 12 and 13, in the absorption spectra of FADGDH solutions, typical FAD-derived peaks (“FADGDH” in FIGS. 12 and 13) were observed. On the other hand, in the absorption spectrum of the FADGDH solution after addition of glucose, a peak derived from FAD (absorption peak of 350 to 500 nm) disappeared (“FADGDH+glucose” in FIGS. 12 and 13). Also in the absorption spectrum of the FADGDH solution after addition of sodium dithionite, a peak derived from FAD disappeared (“FADGDH+sodium dithionite” in FIGS. 12 and 13).

These results reveal that both of the FADGDH species (FADGDH derived from Ta. emersonii and FADGDH derived from Th. crustaceus) expressed in E. coli and purified are purified as holoenzymes containing oxidized FAD as a coenzyme. Therefore, FADGDH derived from thermophilic filamentous fungi of the present invention produced by E. coli has an advantage of FADGDH that it can be used as a glucose concentration measuring reagent or the like without requiring addition of a coenzyme.

### Example 6

In the present example, FADGDH derived from Ta. emersonii was produced by using yeast (Pichia pastoris). Hereinafter, the details will be described.

First, PCR was conducted using a Ta. emersonii-derived FADGDH gene-coding region as a template, and primers for preparation of secretory expression type plasmid, 31232Pic1 (5′-AAAGAATTCGCTCCGCTGTGCCAGACTGCC-3′) and 31232Pic2 (5′ TTTGCGGCCGCTCAGTGGTGGTGGTGGTGGTGATTGCCCAATCTGGCCGCA TC-3′) to amplify the gene encoding matured FADGDH. On the C-terminal side of the amplified gene that encodes Ta. emersonii matured FADGDH, the codon (CAC) for histidine for fusion with a histidine tag is added. The amplified DNA fragment was cut with EcoRI and NotI, and then connected in the equivalent restriction enzyme site of secretory expression vector pPIC9 (Life Technologies) for P. pastoris (Pichia pastoris) to prepare Ta. emersonii-derived FADGDH gene secretory expression type plasmid. By connecting the gene encoding matured FADGDH to pPIC9, a signal sequence for yeast is fused on the upstream of the gene encoding matured FADGDH.

Ta. emersonii-derived FADGDH gene secretory expression type plasmid was introduced into P. pastoris GS115 strain (Life Technologies). The GS115 strain into which the secretory expression type plasmid had been introduced was cultured for 2 days in a BMG culture medium (100 mM potassium phosphate buffer: pH 6.0, 1.34% yeast nitrogen base, 4×10⁻⁵% biotin, 1% glycerol) (pre-culture). The cells obtained by the pre-culture were inoculated in a BMM culture medium (100 mM potassium phosphate buffer: pH 6.0, 1.34% yeast nitrogen base, 4×10⁻⁵% biotin, 0.5% methanol), and aerobically cultured at 30° C. for about 5 days.

The culture supernatant after the culture was collected, and dialyzed against a PBS buffer. The culture supernatant after dialysis was applied to a Ni-NTA Agarose (QIAGEN) column (diameter 2.0 cm, height 2.0 cm) equilibrated with a PBS buffer. After washing the column with a PBS buffer, the adsorbed protein was eluted with 20 mM Hepes-NaOH buffer (pH 7.5) containing 200 mM imidazole. The eluted fraction was subjected to demineralization using Amicon Ultra-4 MWCO10 (Millipore) to give a Ta. emersonii-derived FADGDH purified sample.

### Example 7

In the present example, FADGDH derived from Th. crustaceus was produced by using yeast (Pichia pastoris). Hereinafter, the details will be described.

First, PCR was conducted using a Th. crustaceus-derived FADGDH gene-coding region as a template, and primers for preparation of secretory expression type plasmid, 9129Pic1 (5′-AAAGAATTCACCTCCTATGACTACATTATC-3′) and 9129Pic2 (5′-TTTGCGGCCGCTTAGTGGTGGTGGTGGTGGTGGACCTGACCGGCCTTGATG AG-3′) to amplify the gene encoding matured FADGDH. On the C-terminal side of the amplified gene that encodes Th. crustaceus matured FADGDH, the codon (CAC) for histidine for fusion with a histidine tag is added. The amplified DNA fragment was cut with EcoRI and NotI, and then connected in the equivalent restriction enzyme site of secretory expression vector pPIC9 (Life Technologies) for P. pastoris to prepare Th. crustaceus-derived FADGDH gene secretory expression type plasmid. By connecting the gene encoding matured FADGDH to pPIC9, a signal sequence for yeast is fused on the upstream of the gene encoding matured FADGDH.

Th. crustaceus-derived FADGDH gene secretory expression type plasmid was introduced into P. pastoris GS115 strain (Life Technologies). The GS115 strain into which the secretory expression type plasmid had been introduced was cultured for 2 days in a BMG culture medium (100 mM potassium phosphate buffer: pH 6.0, 1.34% yeast nitrogen base, 4×10⁻⁵% biotin, 1% glycerol) (pre-culture). The cells obtained by the pre-culture were inoculated in a BMM culture medium (100 mM potassium phosphate buffer: pH 6.0, 1.34% yeast nitrogen base, 4×10⁻⁵% biotin, 0.5% methanol), and aerobically cultured at 30° C. for about 5 days.

The culture supernatant after the culture was collected, and dialyzed against a PBS buffer. The culture supernatant after dialysis was applied to a Ni-NTA Agarose (QIAGEN) column (diameter 2.0 cm, height 2.0 cm) equilibrated with a PBS buffer. After washing the column with a PBS buffer, the adsorbed protein was eluted with 20 mM Hepes-NaOH buffer (pH 7.5) containing 200 mM imidazole. The eluted fraction was subjected to demineralization using Amicon Ultra-4 MWCO10 (Millipore) to give a Th. crustaceus-derived FADGDH purified sample.

### Example 8

### SDS-PAGE

As for each FADGDH purified sample obtained in Examples 6 and 7 (FADGDH derived from Ta, emersonii and FADGDH derived from Th. crustaceus expressed in P. pastoris and purified), the protein concentration in each FADGDH purified liquid was measured in the same manner as described in Example 4. Based on this measured concentration value, the purified sample containing 5 μg of FADGDH was subjected to SDS-polyacrylamide electrophoresis (SDS-PAGE).

The SDS-PAGE was conducted according to the method of Laemmli. As SDS-polyacrylamide gel, “e-PAGEL 12.5%” (ATTO Corporation) was used. The gel after migration was stained with a CBB (Coomassie Brilliant Blue)-based stain solution “GelCode Blue Safe Protein Stain” (Thermo Fisher Scientific K.K.). As a protein marker, “Protein Ladder (10-250 kDa)” (New England BioLabs) was used.

SDS-PAGE was conducted in a similar manner except that a PAS (Periodic acid-Schiff stain) stain solution was separately used as a stain solution. The PAS stain solution selectively oxidizes saccharides to generate aldehyde, and turns reddish purple by a Schiff reagent.

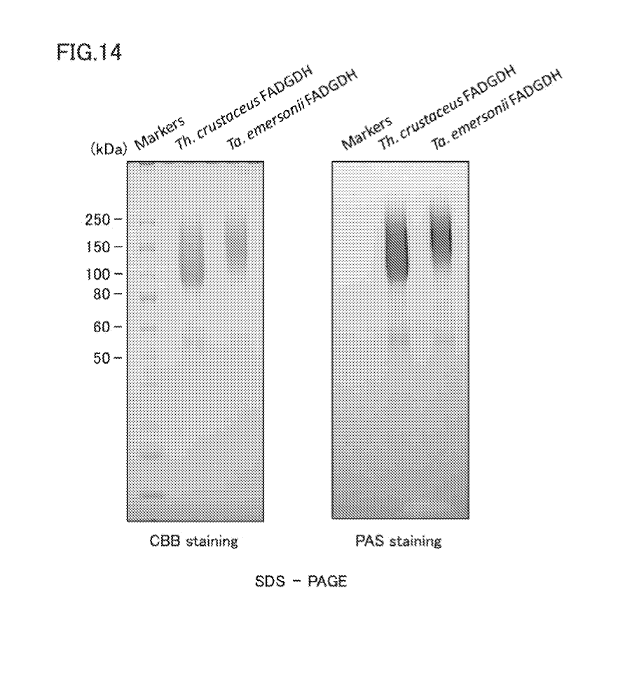

The result of SDS-PAGE is shown in FIG. 14. The left photograph shows the result when the CBB-based stain solution is used, and the right photograph shows the result when the PAS stain solution is used. As shown in FIG. 14 (left photograph), it was revealed that for both of FADGDH derived from Ta. emersonii and FADGDH derived from Th. crustaceus, electrophoretically single purified samples were successfully obtained. Also as shown in FIG. 14 (right photograph), it was revealed that both of these purified samples are glycosylated proteins (FADGDH).

### Example 9

### Measurement of Absorption Spectrum

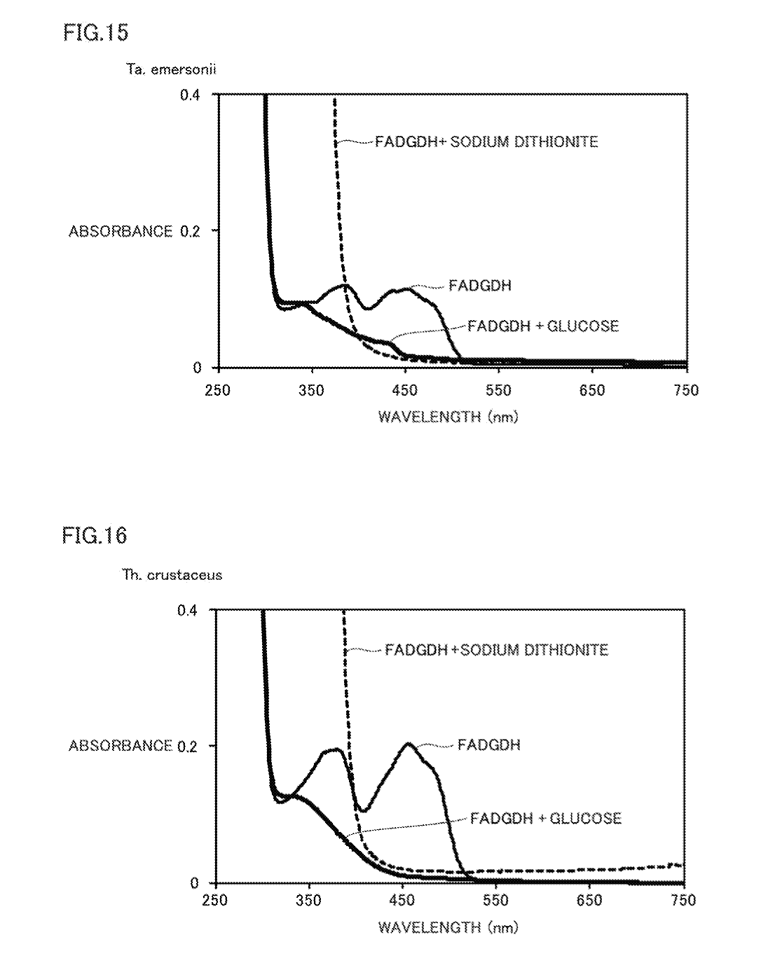

Each FADGDH purified sample obtained in Examples 6 and 7 (FADGDH derived from Ta. emersonii and FADGDH derived from Th. crustaceus expressed in P. pastoris and purified) was diluted based on the measured concentration value in Example 8, and a 2 mg/mL enzyme solution (FADGDH solution) was prepared. An absorption spectrum of the FADGDH solution was measured by using a UV/visible spectrophotometer DU-800 (Beckman Coulter). Also for the solution prepared by adding glucose to the FADGDH solution in a final concentration of 150 mM, and the solution prepared by adding sodium dithionite powder which is a reducing agent to the FADGDH solution, an absorption spectrum was measured in a similar manner. The obtained absorption spectra are shown in FIG. 15 (FADGDH derived from Ta. emersonii) and FIG. 16 (FADGDH derived from Th. crustaceus).

As shown in FIGS. 15 and 16, in the absorption spectra of FADGDH solutions, typical FAD-derived peaks (“FADGDH” in FIGS. 15 and 16) were observed. On the other hand, in the absorption spectrum of the FADGDH solution after addition of glucose, a peak derived from FAD (absorption peak of 350 to 500 nm) disappeared (“FADGDH+glucose” in FIGS. 15 and 16). Also in the absorption spectrum of the FADGDH solution after addition of sodium dithionite, a peak derived from FAD disappeared (“FADGDH+sodium dithionite” in FIGS. 15 and 16).

These results reveal that both of the FADGDH species (FADGDH derived from Ta. emersonii and FADGDH derived from Th. crustaceus) expressed in yeast (P. pastoris) and purified are purified as holoenzymes containing oxidized FAD as a coenzyme. Therefore, FADGDH derived from thermophilic filamentous fungi of the present invention produced by yeast has an advantage of FADGDH that it can be used as a glucose concentration measuring reagent or the like without requiring addition of a coenzyme.

### Example 10

In the present example, for FADGDH purified samples obtained in Examples 2 and 3 (solutions containing Ta. emersonii-derived FADGDH and Th. crustaceus-derived FADGDH expressed in E. coli and purified), and FADGDH purified samples obtained in Examples 6 and 7 (FADGDH derived from Ta. emersonii and FADGDH derived from Th. crustaceus expressed in P. pastoris and purified) (a total of four FADGDH purified samples), evaluation tests for the following various properties regarding the enzyme activity of FADGDH (pH stability, pH dependency, temperature stability, temperature dependency, substrate specificity, responsiveness in glucose sensor chip) were conducted. The principle of enzyme activity measurement, the definition of enzyme activity, and the activity measuring method in the present example are as follows.

[Principle of Enzyme Activity Measurement]

D-glucose 1-methoxyPMS+FADGDH

→D-glucono-1,5-lactone+1-methoxyPMS (reduced form)

+FADGDH (oxidized form)

1-methoxyPMS (reduced form)+DCPIP

→1-methoxyPMS+DCPIP (reduced form)

The amount of reduced form DCPIP that was generated by reduction of 2,6-dichlorophenolindophenol (DCPIP) by reduced form 1-methoxyphenazinemethosulfate (1-methoxyPMS) was determined by measuring the absorbance at 600 nm by using a spectrophotometer. In examination of the substrate specificity, D-glucose was changed to other saccharides, and the enzyme activity for each saccharide (substrate) was measured.

[Definition of Enzyme Activity]

Activity of enzyme (FADGDH activity) is so defined that the amount of FADGDH that allows formation of 1 micromole of reduced form DCPIP per minute under the conditions of 37° C., pH 7.0 is 1 unit (U).

Concretely, FADGDH activity can be determined from the later-described change in the absorbance (ΔOD_{TEST}, ΔOD_{BLANK}) according to the following formula.

FADGDH activity (U/mL)=[(ΔOD_{TEST}−ΔOD_{BLANK})×3.1]/(16.3×0.1×dilution ratio)

The constants in the formula have the following meanings.

3.1: Volume of reaction liquid after mixing of FADGDH solution (mL)

16.3: Millimolar molecular extinction coefficient of DCPIP (mM⁻¹·cm⁻¹)

0.1: Volume of FADGDH solution (mL)

[Activity Measuring Method]

(1) Mix the following ingredients of the reaction liquid in a cuvette.

0.1 M potassium phosphate buffer (pH 7.0) 1.5 mL

1 M glucose solution 0.9 mL

1.75 mM 2,6-Dichlorophenolondophenol (DCPIP) 0.12 mL

20 mM 1-Methoxy-5-methylphenazium methylsulfate (1-mPMS) 0.021 mL

10% TritonX-100 0.06 mL

H₂O 0.399 mL

(2) Pre-incubate the reaction liquid at 37° C. for 10 minutes.

(3) Add 0.1 mL of a FADGDH solution and mix the solution by inversion, and allow the mixture to react at 37° C. During this term, record the absorbance at 600 nm by using an absorptiometer, and calculate the change in the absorbance per minute (ΔOD_{TEST}).

(4) As a reference, for an equivalent amount of a solution for diluting enzyme (solution prepared by adding 0.01% TritonX-100 to 50 mM phosphate buffer (pH 7.0)) in place of the enzyme liquid (the liquid prepared by adding a FADGDH solution to the aforementioned reaction liquid), record the variation of the absorbance and calculate the change in the absorbance per minute (ΔOD_{BLANK}) in a similar manner as in (3).

<pH Stability>

Here, pH stability of the enzyme activity was evaluated. For each of the four purified samples including the FADGDH purified samples obtained in Examples 2 and 3 (solutions containing Ta. emersonii-derived FADGDH and Th. crustaceus-derived FADGDH expressed in E. coli and purified), and FADGDH purified samples obtained in Examples 6 and 7, the enzyme activity was measured by the aforementioned activity measuring method. By diluting each purified sample in water based on the measured value, an enzyme solution having an enzyme activity of 14 U/mL (FADGDH solution) was prepared.

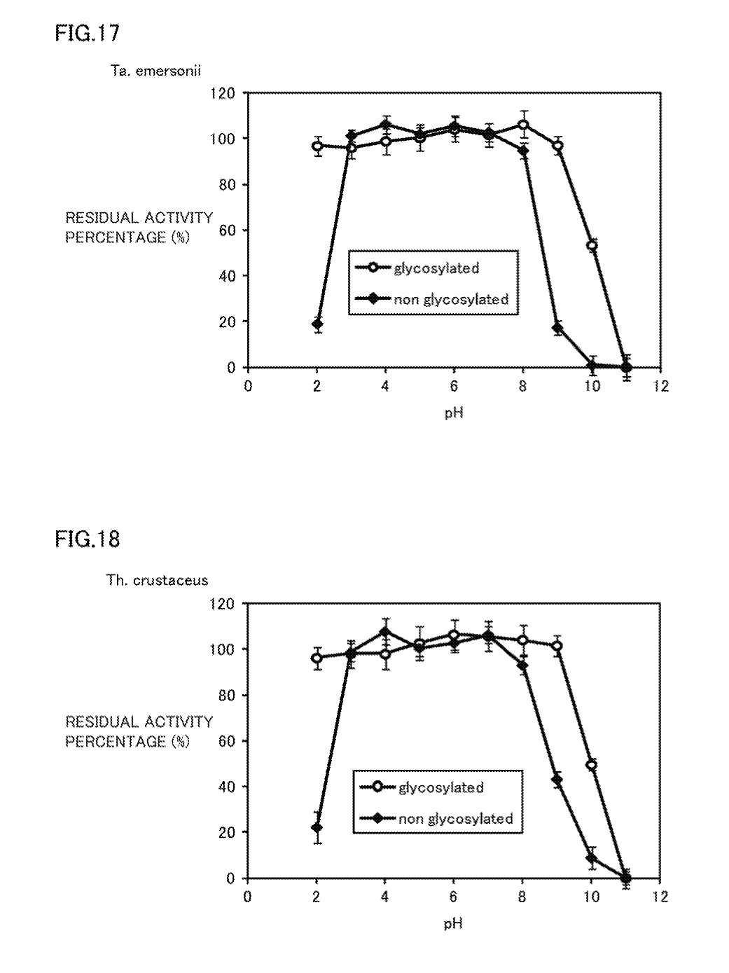

For these FADGDH solutions, an equivalent amount of 0.1 M Britton-Robinson universal buffer having a pH adjusted to a predetermined value was mixed, to prepare a plurality of enzyme solutions having a pH varying in the range of 2.0 to 11.0, and enzyme activity immediately after preparation of each enzyme solution was measured. Next, after incubating each sample solution at 25° C. for 20 hours, the enzyme activity was measured, and the percentage of the enzyme activity after incubation to the enzyme activity before incubation (residual activity percentage) was determined. The results are shown in FIG. 17 (FADGDH derived from Ta. emersonii) and FIG. 18 (FADGDH derived from Th. crustaceus).

In FIG. 17, in the case of FADGDH derived from Ta. emersonii, the one that was expressed in E. coli and was not glycosylated (hereinafter referred to as “non-glycosylated”) was stable at pH 3.0 to 7.0, and exhibited a residual activity percentage of more than or equal to 90% at pH 7.0 to 8.0. The one that was expressed in yeast and glycosylated (hereinafter referred to as “glycosylated”) was stable over the wide range of pH from 2.0 to 9.0. Also in FIG. 18, in the case of FADGDH derived from Th, crustaceus, almost the same tendency with the case of FADGDH derived from Ta. emersonii was shown.

<pH Dependency>

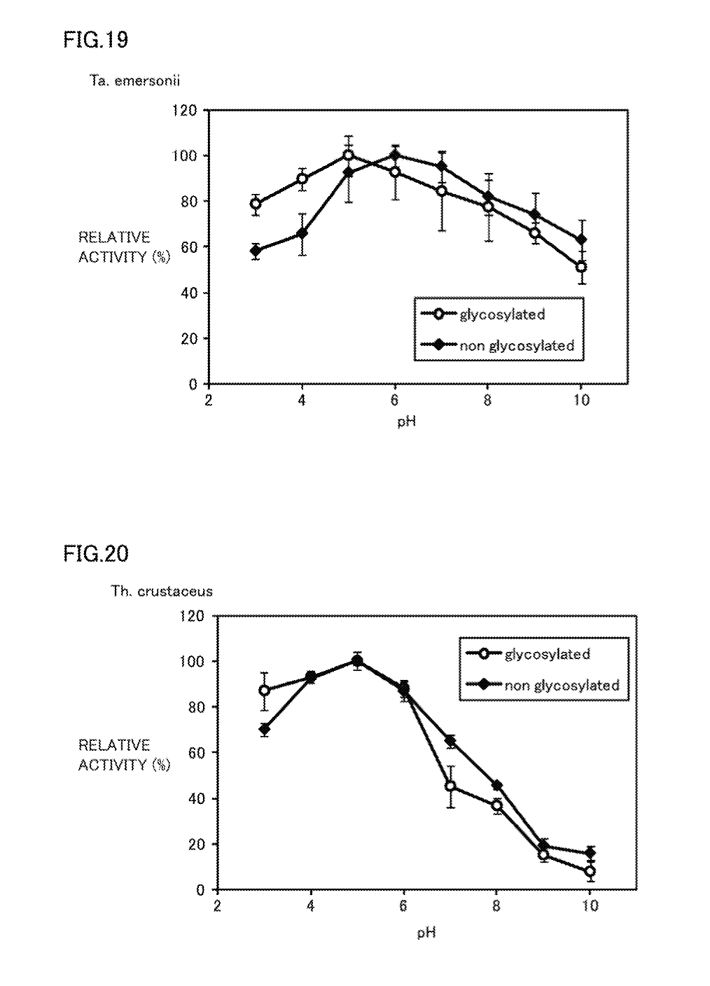

Here, pH dependency of the enzyme activity was evaluated. In a similar manner to the evaluation test of pH stability, enzyme solutions (FADGDH solutions) were prepared so that the enzyme activity was 0.2 U/mL. A plurality of enzyme activity measuring solutions having various pH values in the range of 3.0 to 10.0 were prepared, and the enzyme activity at each pH condition was measured. Letting the enzyme activity in the pH condition where the activity was maximum be 100(%), the percentage of enzyme activity in each pH condition was determined as the relative activity. The results are shown in FIG. 19 (FADGDH derived from Ta. emersonii) and FIG. 20 (FADGDH derived from Th. crustaceus).

In FIG. 19, it was proved that the optimum pH of FADGDH derived from Ta. emersonii was pH 5.0 in “glycosylated” FADGDH and pH 6.0 in “non-glycosylated” FADGDH and a relative activity of more than or equal to 50% was kept over a wide range of pH from 3.0 to 10.0. In FIG. 20, FADGDH derived from Th. crustaceus showed the highest activity at pH 5.0 regardless of whether it was glycosylated or non-glycosylated, and was shown to have high enzyme activity in the acidic region.

<Temperature Stability>

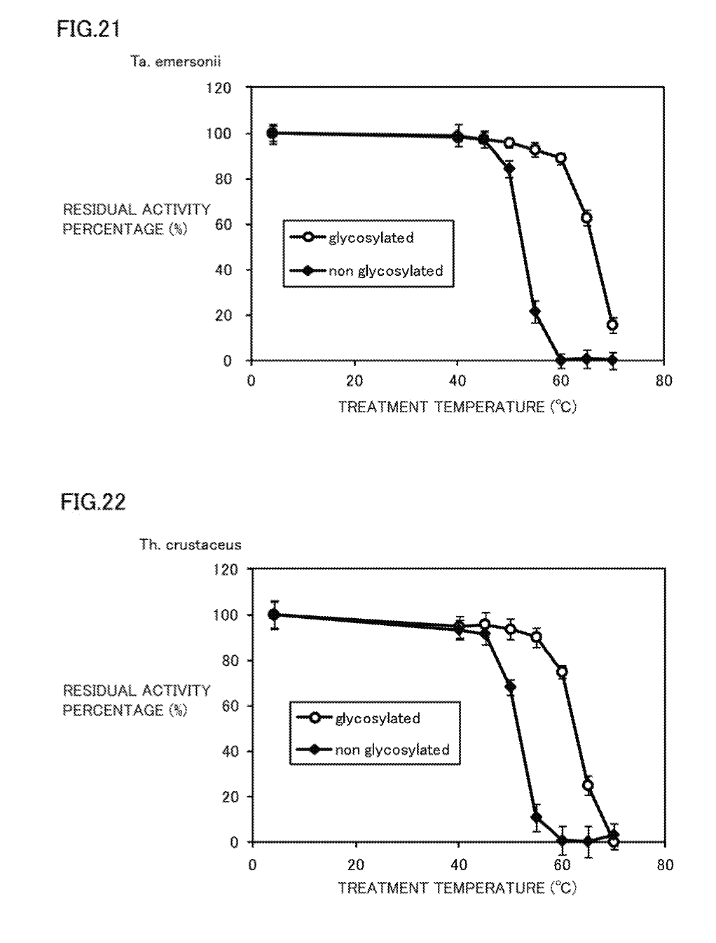

Here, temperature stability of the enzyme activity was evaluated. In a similar manner to the evaluation test of pH stability, enzyme solutions (FADGDH solution) were prepared so that the enzyme activity was 7 U/mL. These FADGDH solutions were dispensed into microtubes, and heated for 15 minutes at several temperatures ranging from 40° C. to 70° C. using a PCR machine. The enzyme activity was measured before and after heating, and the percentage of the enzyme activity after heating to the enzyme activity before heating (residual activity percentage) was determined. The results are shown in FIG. 21 (FADGDH derived from Ta. emersonii) and FIG. 22 (FADGDH derived from Th. crustaceus).

In FIG. 21, FADGDH derived from Ta. emersonii (glycosylated) kept about 90% of the activity even at 60° C., and was shown to have very high thermal stability. FADGDH derived from Ta. emersonii (non-glycosylated) almost lost the activity at 60° C., however, it showed very high stability at less than or equal to 45° C., and kept more than or equal to 80% of the activity even at 50° C. Further, in FIG. 22, FADGDH derived from Th. crustaceus (glycosylated) kept nearly 80% of the activity even at 60° C., and was shown to have very high thermal stability. FADGDH derived from Th. crustaceus (non-glycosylated) almost lost the activity at 60° C., however, it showed very high stability at less than or equal to 45° C., and kept nearly 70% of the activity even at 50° C. Since naturally occurring Th. crustaceus and Ta. emersonii are eukaryotes that involve glycosylation, it is presumed that the naturally occurring enzymes also show high thermal stability.

These results indicate that the protein having FADGDH activity of the present invention has very high thermal stability, particularly when it is glycosylated. It is also presumed that the one that is not glycosylated has sufficient thermal stability in such a temperature environment in ordinary distribution, transportation and so on.

<Temperature Dependency>

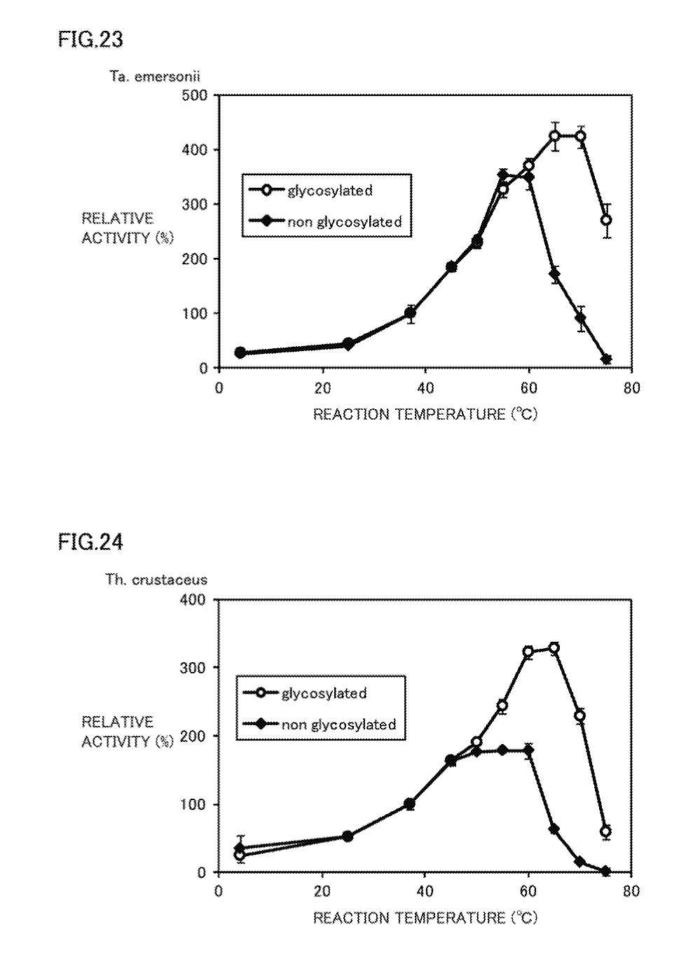

Here, temperature dependency of the enzyme activity was evaluated. First, in a similar manner to the evaluation test of pH stability, enzyme solutions (FADGDH solutions) were prepared so that the enzyme activity was 0.05 to 0.2 U/mL. For these FADGDH solutions, the enzyme activity was measured at temperatures ranging from 4° C. to 75° C., and the relative activity to the activity at 37° C. of 100% was determined. The results are shown in FIG. 23 (FADGDH derived from Ta. emersonii) and FIG. 24 (FADGDH derived from Th. crustaceus).

As shown in FIG. 23, in the case of FADGDH derived from Ta. emersonii, the activity peaked at 55° C. to 60° C. and decreased at higher temperatures in the case of “non-glycosylated” FADGDH. The “glycosylated” FADGDH showed substantially the same activity as “non-glycosylated” FADGDH up to 55° C., however the activity increased at higher temperatures, and the activity peaked at 65° C. to 70° C. and then decreased at higher temperatures. Also as shown in FIG. 24, in the case of FADGDH derived from Th. crustaceus, the activity peaked at 45° C. to 60° C. and decreased at higher temperatures in the case of “non-glycosylated” FADGDH. The “glycosylated” FADGDH showed substantially the same activity as “non-glycosylated” FADGDH up to 45° C., however the activity increased at higher temperatures, and the activity peaked at 60° C. to 65° C. and then decreased at higher temperatures. The “glycosylated” FADGDH showed an activity of more than or equal to three times in Th. crustaceus and more than or equal to four times in Ta. emersonii at the peak compared with the activity at 37° C.

<Substrate Specificity>

Here, whether saccharides other than glucose can be used as a substrate was examined. Each enzyme solution (FADGDH solution) was prepared to have an enzyme concentration with which a sufficient enzyme activity would be obtained at a glucose concentration of 40 mM. For these FADGDH solutions, the enzyme activity was measured by the aforementioned activity measuring method while the concentration of saccharides in the reaction liquid was changed to 40 mM. Measurement was conducted four 14 kinds of saccharides shown in Table 2. A sample not containing enzyme was prepared as a control, and a control was prepared for each kind of saccharide. The relative activity to 100% of the measured value of enzyme activity for glucose was calculated for each saccharide, and collectively shown in Table 2. A sample exhibiting no significant difference in variation of DCPIP absorbance compared with the control was determined as not having activity, as denoted by 0.0.

TABLE 2

| | Ta. emersonii | | Th. crustaceus | |
|---|---|---|---|---|
| Substrate | | non- | | non- |
| (40 mM) | glycosylated | glycosylated | glycosylated | glycosylated |
| D-Glucose | 100 | 100 | 100 | 100 |
| D-Arabinose | 0.0 | 0.0 | 0.0 | 0.0 |
| D-Fructose | 0.0 | 0.0 | 0.0 | 0.0 |
| D-Galactose | 0.0 | 0.0 | 0.0 | 0.0 |
| 2-deoxy- | 11.5 | 17.7 | 60.1 | 49.8 |
| D-Glucose | | | | |
| Maltose | 0.0 | 0.0 | 0.0 | 0.0 |
| Maltotetraose | 0.0 | 0.0 | 0.0 | 0.0 |
| Maltotriose | 3.4 | 2.7 | 0.0 | 0.0 |
| D-Mannitol | 0.0 | 0.0 | 0.0 | 0.0 |
| D-Mannose | 0.0 | 0.0 | 6.8 | 5.6 |
| D-Raffinose | 0.0 | 0.0 | 0.0 | 0.0 |
| D-Sorbitol | 0.0 | 0.0 | 0.0 | 0.0 |
| Sucrose | 0.0 | 0.0 | 0.0 | 0.0 |
| D-Trehalose | 0.0 | 0.0 | 0.0 | 0.0 |
| D-Xylose | 24.8 | 24.6 | 27.6 | 21.7 |

As shown in the result of Table 2, both FADGDH derived from Ta. emersonii and FADGDH derived from Th. crustaceus failed to react with D-arabinose, D-fructose, D-galactose, maltose, maltotetraose, D-mannitol, D-raffinose, D-sorbitol, sucrose and D-trehalose among the 14 kinds of examined saccharides. Also for maltotriose, FADGDH derived from Th. crustaceus failed to react, and FADGDH derived from Ta. emersonii merely slightly reacted. Also for D-mannose, FADGDH derived from Ta. emersonii failed to react, and FADGDH derived from Th. crustaceus merely slightly reacted. For 2-deoxy-D-glucose and D-xylose, both of the FADGDH species recognized as a substrates, however, this compound is not an ingredient contained in medical transfusion like maltose, and even if it is present in blood, the quantity is negligibly small in comparison with the quantity of glucose, and hence it will not constitute a hindrance in use in a glucose sensor or the like.

<Responsiveness in Glucose Sensor Chip>

An electrode base was prepared by disposing a working electrode and a counter electrode on an insulating substrate, and a reactive layer was defined by using an adhesive layer. On the surface of the electrode, a recombinant FADGDH purified sample as described above and a mediator (potassium ferricyanide) were applied, and naturally dried, and a cover layer was overlapped for individualization to form a sensor chip. Four chips respectively using the four recombinant FADGDH species were prepared.

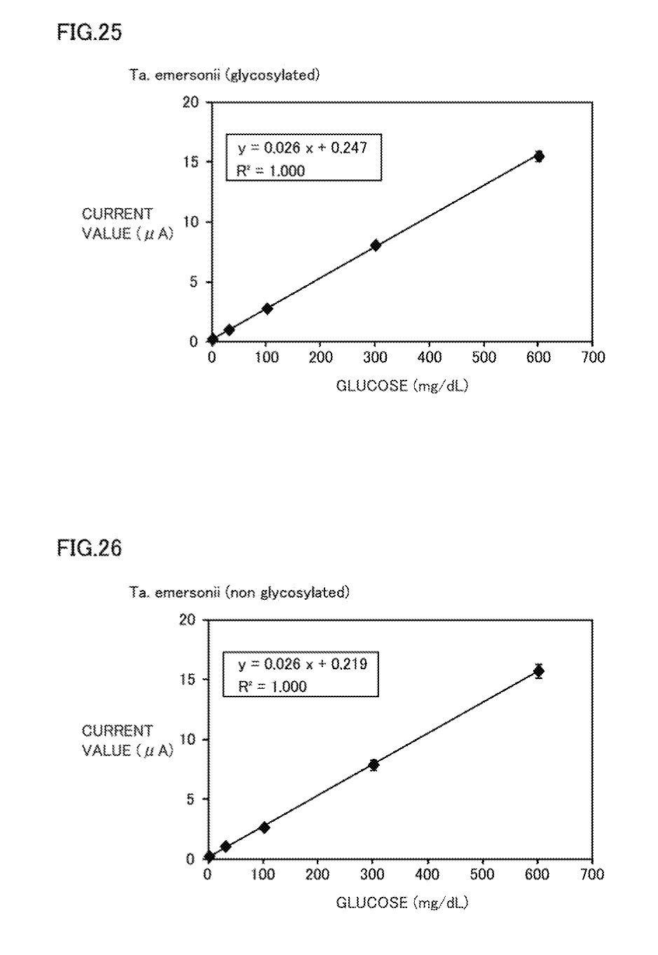

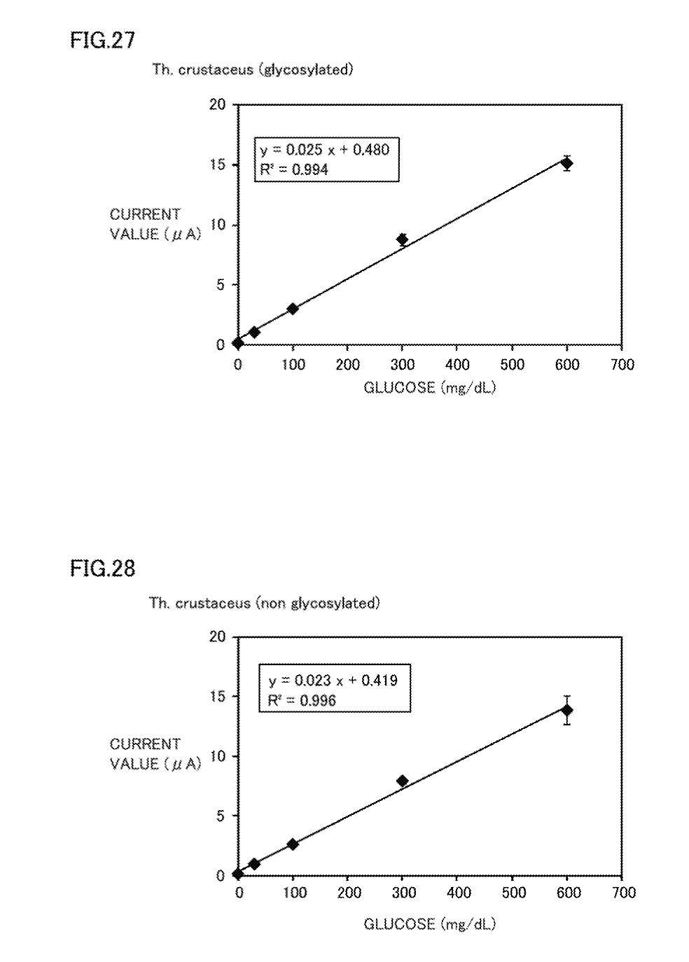

Solutions containing 0 to 600 mg/dL glucose in potassium phosphate buffer at pH 7.4 were prepared, the current value arising at the time when 1 μL of the glucose solution was introduced into the chip from a sample inlet was measured, and each measured value was plotted against the glucose concentration. For the plots, a regression line was drawn in the range of 0 to 600 mg/dL by the least square method. The results are shown in FIG. 25 (FADGDH derived from Ta. emersonii: glycosylated), FIG. 26 (FADGDH derived from Ta. emersonii: non-glycosylated), FIG. 27 (FADGDH derived from Th. crustaceus: glycosylated), and FIG. 28 (FADGDH derived from Th. crustaceus: non-glycosylated).

In FIG. 25 to FIG. 28, R² (coefficient of determination: square of correlation coefficient R) is 1.000, 1.000, 0.994, and 0.996, respectively, which are nearly 1, and high linearity is shown. Therefore, it is suggested that the glucose concentration can be quantified with high accuracy by using FADGDH derived from Ta. emersonii and FADGDH derived from Th. crustaceus regardless of whether it is expressed in E. coli or it is expressed in yeast.

1. A protein derived from a thermophilic filamentous fungus, having flavin adenine dinucleotide-dependent glucose dehydrogenase activity.

2. A protein of any one of the following (a) to (d):
(a) a protein having an amino acid sequence represented by SEQ ID NO: 1 or 2,
(b) a protein having an amino acid sequence that is the amino acid sequence represented by SEQ ID NO: 1 or 2 from which a signal peptide is removed,
(c) a protein having an amino acid sequence that is the amino acid sequence of the protein of the above (a) or (b) from/to which one or several amino acid residues are deleted, substituted, added or inserted, and
(d) a protein having a homology of more than or equal to 70% with the amino acid sequence of the protein of any one of the above (a) to (c), and having flavin adenine dinucleotide-dependent glucose dehydrogenase activity.

3. A protein comprising an amino acid sequence encoded by at least one base sequence selected from the group consisting of base sequences of SEQ ID NOs: 5 to 7, and base sequences complementary to base sequences of SEQ ID NOs: 8 or 9, and having flavin adenine dinucleotide-dependent glucose dehydrogenase activity.

4. The protein according to claim 2 derived from a thermophilic filamentous fungus.

5. The protein according to claim 1, wherein said thermophilic filamentous fungus is Talaromyces emersonii or Thermoascus crustaceus.

6. The protein according to claim 1, that is glycosylated.

7. A gene encoding the protein according to claim 1.

8. A gene comprising a DNA of any one of the following (A) to (D):
(A) a DNA having a base sequence according to SEQ ID NO: 3 or 4,
(B) a DNA having a base sequence that is the base sequence of SEQ ID NO: 3 or 4 from which a base sequence encoding a signal peptide is removed,
(C) a DNA containing the base sequence of the DNA of (A) or (B) and an intron, and
(D) a DNA that hybridizes with a DNA having a base sequence complementary to the DNA of any one of (A) to (C) under a stringent condition, and encodes a protein having flavin adenine dinucleotide-dependent glucose dehydrogenase activity.

9. A recombinant vector comprising the gene according to claim 8.

10. A transformant comprising the gene according to claim 8.

11. A method for producing flavin adenine dinucleotide-dependent glucose dehydrogenase using the gene according to claim 8.

12. A method for producing flavin adenine dinucleotide-dependent glucose dehydrogenase using the transformant according to claim 10.

13. A glucose concentration measuring reagent comprising the protein according to claim 1.

14. A glucose sensor using the protein according to claim 1.

15. A glucose concentration measuring method using the protein according to claim 1.

16. A biofuel cell using the protein according to claim 1.


SEQUENCE LISTING (9 sequences)

SEQ ID NO: 1 - protein, 593 aa, Talaromyces emersonii
MLRRTVGILAVLSLASAAPLCQTASSSYDYIVVGGGTSGLVVANRLSENPNVSVLVIEAGDSVYNNANVTNVNGYGLAFGTPIDWQYQSTNQTYAGNTRQTLRAGKALGGTSTINGMAYTRAQDVQIDAWAAIGNDGWDWSSLWPYYLKSEAFTAPNQTQRAAGASYNPAYHGVTGPLHVGFIEMQPNNLSSILNQTYQALGVPWTEDVNGGKMRGYNFFPSTVDDAADVREDAARAYYYPFESRPNLRVMLNTLANRIVWKNETSGGNVTADGVEVTPLNGTVCRIQANNEVILSAGSLRSPGILELSGVGNPSILNKYNIPVKVNLPTVGENMQDQMYNDASAEGYSAIAGTKSVAYPSVTDLFGNRTSAVAASVQNQLAQYAEAAANQSQGTMKASDLQRLFQIQYDLIFKQEVPIAEIITYPTGNTLAAGYWGLLPFARGSVHIASADPTVQPVINPNYFMFDWDVQQQIGTAKFIRNLYKTAPLSSLVKNETEPGSAVPEGASDSVWEAWLKETYRSNFHPVGTAAIMPRSIGGVVDERLRVYGTANVRVVDASVLPFQICGHLTSTLYAVAERASDFLKEDAARLGN

SEQ ID NO: 2 - protein, 584 aa, Thermoascus crustaceus
MLLPLSLLSLCSVAAATSYDYIIVGGGTSGLVVANRLSEDSSVSVLVVEAGDSVYNNPNVTGTGAYGAAFGTSIDWAYQTIKQPYAGDLTQTIRAGKALGGTSTINGMAYTRAEDVQIDAWEAIGNQGWNWSTLFPYYLKSERFQIPINAQSAAGANWNPAYHGMNGPLTTGWTYEMMNGSMAEVLNATYQNLGISYLPDVNGGKMHGYTLFPRTVNRALDVREDAARAYYYPFESSRPNLSVMLNTYGNRIVWANATNTNSVTATGVEVTLSDGTVQTIQANKEVILSAGSLISPAILERSGVGNPTVLSRFGIPLVVNLTTVGENLQDQMNTEFIYLSNATFSGQSTFLGHPTAADLFGSNVTNVAKSVQSSLPHYAATVSEASNGTMSAAVLQKLFQLQYNIIFNNPTPIAEILVAPSGSKFITEYWALLPFARGNVHIGSANPLDRPVINPNYMMFEWDMQEQIASGKFVRKLYGTAPFTNWATGEATPGYSTLPANATDGQWAQWISSVSRSNFHPVGTAAMMPRDMGGVVDSNLVVYGTNNVRVIDASVLPLQVCGHLVSTLYAVAERASDLIKAGQV

SEQ ID NO: 3 - DNA, 1782 bp, Talaromyces emersonii
atgctgcgccggaccgttggcatcctcgcggtgctgtccctggcgtctgcagctccgctgtgccagactgcctccagctcgtatgactacatcgtcgtcggcggcggcaccagcggtctggtcgtcgcaaaccgcctgtccgaaaatccgaacgtgtccgtcctggtcatcgaggccggcgactcggtctacaacaatgccaacgtgaccaacgtcaatggctacggactggcctttgggacgcccatcgactggcagtaccaatccaccaaccagacctacgctggcaacacgcggcagacgctccgtgctggcaaagccctgggaggcacgagcacgatcaatggaatggcctatacgcgcgcccaagacgtccagatcgatgcctgggcggcgatcgggaatgacggctgggactggagctcgctctggccgtactatctcaagagcgaggcattcaccgcgccgaaccagacgcaacgggcagccggtgcttcttacaacccagcgtaccacggagtgaccggacctctgcatgtgggcttcatcgagatgcagcccaacaacctgtcttcgattctgaaccagacctaccaggctctcggggttccgtggacggaagacgtcaacggaggaaagatgcgtggctacaacttcttcccgtcgacggtcgacgatgcagcagatgttcgggaagatgctgctcgtgcttactactacccctttgagtcccgcccgaatctgagggtcatgctgaacacgctcgcgaaccggattgtctggaagaatgagaccagcggcggaaatgtcactgcggacggagtggaagtcactccgctcaatgggaccgtctgtaggattcaggcaaacaacgaggtcattctgtcggctggttctcttcgctcgccggggattttggaactctctggggttgggaatccaagcattctgaacaaatacaacattcccgtcaaagtcaacctgccaacggtgggagagaacatgcaagaccagatgtacaatgatgccagcgccgagggatattcggccatcgccggtaccaagtctgtcgcctatccatctgtgacggacctctttggcaaccgcaccagcgctgtcgcggcctctgttcagaaccaactggcccagtacgccgaagcagcggccaaccagagccagggaaccatgaaagcgtccgacctgcagcgcctgttccagatccaatatgacctgatcttcaagcaagaggttcccattgccgagatcatcacttatcccaccgggaacaccctggcggccgggtactggggtctccttccctttgctcggggaagcgtgcatatcgcatccgccgacccaacggtccagccggtaataaatcccaattatttcatgttcgactgggatgtgcagcagcagattggcacggccaagtttatccggaacttgtacaagacggcgccgttgagcagtcttgtcaaaaacgaaacggagccaggcagtgctgtgcctgagggagcatcggatagtgtttgggaggcgtggctgaaggagacatatcggtcgaatttccatcccgtcggcaccgcggccataatgccccggtctatcggaggggtggttgacgaacgactgcgggtgtacggcacagcgaatgtgcgcgtggtcgatgcttcggtgcttccattccagatttgcggccatctgaccagcacgctgtatgcagttgcagagcgggcatccgatttcttgaaggaggatgcggccagattgggcaattga

SEQ ID NO: 4 - DNA, 1755 bp, Thermoascus crustaceus
atgttgctccccctttccctcctctccctctgctccgtggccgcggccacctcctatgactacattatcgtcggaggaggcacctcgggtttggtcgtggccaaccgcctgtccgaggactcgtcggtctctgtcctggttgtcgaggctggtgactcggtctacaacaaccccaatgtcaccggcaccggtgcctacggtgcggccttcggcacctccatcgactgggcgtaccagaccatcaaacagccctacgccggagacctcacccagaccatccgtgctggaaaggcccttggaggcaccagcaccatcaacggcatggcctatacccgtgcggaggacgtccagatcgatgcctgggaggccattggcaaccagggctggaactggtcgactctattcccttactacctgaagagtgagcggttccagatacccatcaacgcccagtctgctgccggtgccaactggaacccggcgtaccacgggatgaacggcccgttgacgactggctggacctacgagatgatgaacggcagcatggccgaagtcctcaacgccacttaccagaacctgggtatttcttacctccccgatgtcaacgggggcaagatgcacgggtacaccctcttcccccgaaccgtcaaccgggccctcgacgtgcgtgaggatgccgccagggcgtactactacccgttcgaatcctccaggcccaatctctcggtcatgctcaacacctatggcaaccgcatagtctgggctaacgccaccaacaccaactctgtgactgctactggtgtggaggtcactctcagcgacggtaccgttcaaacaatccaggccaacaaggaggtcatcctctctgccggttctctgatttcaccagcgattctggagcgctctggtgttggtaacccaactgtcctcagccgcttcggcattccccttgttgttaacctgaccaccgtgggtgaaaatctccaggaccagatgaacaccgagttcatctacctgagcaacgctactttcagcggccagtcaaccttcctcggccacccgaccgcggccgacctctttggcagcaacgtgacaaacgtggccaagtccgttcagagcagcctgcctcactatgccgccaccgtgtccgaggccagcaacggtaccatgagcgctgctgttctccagaaattgttccaactgcagtacaacatcatcttcaacaacccgacccccatcgccgagatcttggtcgccccttctggctccaaattcatcactgagtactgggccctgcttccctttgctcgtggaaacgtccacattggctctgccaaccctctggatcgacctgtcatcaaccccaactacatgatgttcgagtgggacatgcaggagcagattgcctcgggcaagtttgtccgcaagctctacgggacagctccgttcaccaattgggctacgggcgaggctactcccgggtactccaccctccctgccaacgccaccgatggccagtgggctcagtggatcagctctgtctctcgttccaacttccaccccgtcggtaccgccgccatgatgcccagggacatgggtggtgttgtcgacagcaacctcgtggtctacggcaccaacaacgtgcgtgtgattgatgcctctgtccttcccttgcaagtctgtggccacttggttagtaccctctacgccgttgcagagcgcgcctccgacctcatcaaggccggtcaggtctaa

SEQ ID NO: 5 - DNA, 26 bp, Artificial
  Oligonucleotide primer designed for PCR
taygaytayatcgttytkggaggcgg

SEQ ID NO: 6 - DNA, 29 bp, Artificial
  Oligonucleotide primer designed for PCR
caagtkctncgtgcrggraaggcccttgg

SEQ ID NO: 7 - DNA, 23 bp, Artificial
  Oligonucleotide primer designed for PCR
acscgcgcmgaggatgtccagat

SEQ ID NO: 8 - DNA, 33 bp, Artificial
  Oligonucleotide primer designed for PCR
catcatggcagcmgtkccgacgggrtggaagtt

SEQ ID NO: 9 - DNA, 29 bp, Artificial
  Oligonucleotide primer designed for PCR
gtgctmaccaartggccgcaracctggaa